(19) United States
(12) Patent Application Publication
(10) Pub. No.: US 2022/0135994 A1
(43) Pub. Date: May 5, 2022

(54) SUPPRESSION OF TARGET GENE EXPRESSION THROUGH GENOME EDITING OF NATIVE MIRNAS

(71) Applicant: SYNGENTA CROP PROTECTION AG, Basel (CH)

(72) Inventors: Juntao LIU, Beijing (CN); Jianping XU, Beijing (CN); Yanhui CHEN, Beijing (CN); Zhiqiang LIU, Beijing (CN); Xi CHEN, Beijing (CN)

(73) Assignee: SYNGENTA CROP PROTECTION AG, Basel (CH)

(21) Appl. No.: 17/432,559

(22) PCT Filed: Feb. 26, 2020
(86) PCT No.: PCT/EP2020/055028

§ 371 Date: Aug. 20, 2021

(30) Foreign Application Priority Data
Mar. 1, 2019 (CN) PCT/CN2019/076722

(51) Int. Cl. C12N 15/82; C12N 15/10; C12N 15/113

(52) U.S. Cl. CPC C12N 15/8278; C12N 15/102; C12N 2310/141; C12N 15/8213; C12N 15/8283; C12N 15/1131

(57) ABSTRACT

The present invention relates to methods and compositions for reducing or suppressing target gene expression by genome editing of native miRNAs

A Sequence Listing in ASCII text format, submitted under 37 C.F.R. § 1.821, entitled “81815_ST25.txt”, 47 kilobytes in size, generated on Feb. 26, 2019. This

Sequence Listing is hereby incorporated by reference into the specification for its disclosures.

### FIELD OF THE INVENTION

The present invention relates to methods and compositions for reducing or suppressing target gene expression by genome editing of native miRNAs.

### BACKGROUND OF THE INVENTION

MicroRNAs (miRNAs), transcribed and processed from longer RNAs (pre-miRNA) that contain imperfect hairpins, are RNAs of about 20-24 nucleotides. miRNAs can precisely target and reduce or suppress the expression of its mRNA target gene in a post-transcriptional manner (Yu et al. 2017, New Phytol. Volume 216(4), pages 1002-1017; Gebert and MacRae 2019, Nature Reviews Molecular Cell Biology, volume 20, pages 21-37). miRNA mediated gene expression inhibition is highly specific and effective compared with small interfering RNA induced RNAi. miRNAs have been used e.g. to target exogenous RNAs from pathogens through transgenic approaches (e.g. WO2010/123904) whereby the artificial miRNA is ectopically over-expressed. This approach can be efficacious; however, relying on genetic transformation of plants requires a high number of transformation events to identify events which show a good expression level while preserving the agronomic characteristics and advantages of the recipient plant. Furthermore, these events are considered to be genetically modified organisms (GMOs) which either are banned from commercialization, or must undergo costly and lengthy regulatory processes to get to the market place.

Consequently, there is a need for improving methods relying on the use of miRNAs to regulate target gene expression.

### SUMMARY

This disclosure provides a novel target gene silencing method using genome editing to swap the 20-24 nucleotide long native miRNA core embedded in the native pre-miRNA with an amiRNA core sequence derived from and designed to be complementary to the target gene sequence. The modification of the native pre-miRNA will generate alternative artificial miRNAs specific to additional target gene transcripts, thereby conferring novel phenotypes, e.g. novel resistances against pests such as viruses.

The present invention provides a method of reducing expression of a target gene comprised of, introducing into a plant cell a nuclease capable of site-directed DNA cleavage at a genomic site encoding a native pre-miRNA of said plant cell, making at least one double strand break at said genomic site or in the vicinity of said genomic site, selecting for a cell where said at least one double strand break has been repaired with an intervening DNA replacing said genomic site, and reducing expression of the target gene, wherein said intervening DNA encodes a modified pre-miRNA comprising an amiRNA core sequence complementary to said target gene.

Amongst other advantages, this method relying on genome editing technology to precisely and specifically reprogram native pre-miRNAs to be complementary to different target genes can lead to the generation of plants which can be considered GMO-free, since limited to no foreign DNA is left in the plant genome after the method is carried out.

Another advantage of this method relies on the ability to generate plants which have one copy of the native miRNA and one copy of the modified/edited miRNA at the same locus. This is in particular relevant to hybrid crops which can then express the newly modified miRNA copy to target a different gene of interest, while retaining a copy of the native mRNA and its associated biological function. An additional benefit compared to previous approaches relying on genetic transformation lies on the fact that the resulting edited plant cell bears one copy of each miRNA (one copy of the native miRNA and one copy of the amiRNA) whereas a plant cell obtained according to prior art methods bears two copies of each version of the miRNA (two copies of the native miRNA and two copies of the amiRNA), which is more demanding for plant cell metabolism and can potentially affect plant performance.

In a further embodiment, the invention relates to the method of the preceding embodiment wherein the target gene is an exogenous target gene, more preferably a pest gene, more preferably a viral, fungal or microbial gene.

In a further embodiment, the invention relates to the method of any one of the preceding embodiments, wherein the target gene is a Bunyavirales gene, preferably a tospovirus gene, more preferably a tomato spotted wilt virus (TSWV) gene.

In a further embodiment, the invention relates to the method of any one of the preceding embodiments, wherein the target gene is an endogenous plant gene.

In a further embodiment, the invention relates to the method of any one of the preceding embodiments, wherein the target endogenous plant gene is a gene involved in plant development, biotic or abiotic stress.

In a further embodiment, the invention relates to the method of any one of the preceding embodiments, wherein said plant cell is a Solanaceae, corn, rice, canola, soybean or sunflower cell. In a further embodiment, the invention relates to the method of any one of the preceding embodiments, wherein said plant cell is a tomato cell.

In a further embodiment, the invention relates to the method of any one of the preceding embodiments, wherein said genomic site encoding a native pre-miRNA encodes a native tomato pre-miRNA.

In a further embodiment, the invention relates to the method of any one of the preceding embodiments, wherein said genomic site comprises SEQ ID NO:6 or SEQ ID NO:7.

In a further embodiment, the invention relates to the method of any one of the preceding embodiments, wherein said intervening DNA comprises any one of SEQ ID NOs: 1 to 5.

In a further embodiment, the invention relates to the method of any one of the preceding embodiments, wherein said nuclease is selected from the group consisting of meganucleases (MNs), zinc-finger nucleases (ZFNs), transcription-activator like effector nucleases (TALENs), Cas9 nuclease, Cfp1 nuclease, dCas9-Fokl, dCpf1-Fokl, chimeric Cas9/Cpf1-cytidine deaminase, chimeric Cas9/Cpf1-adenine deaminase, chimeric FEN1-Fokl, and Mega-TALs, a nickase Cas9 (nCas9), chimeric dCas9 non-Fokl nuclease and dCpf1 non-Fokl nuclease.

In a further embodiment, the invention relates to the method of any one of the preceding embodiments, wherein said cell has a haploid, diploid, polyploid, or hexiploid genome.

In a further embodiment, the invention relates to the method of any one of the preceding embodiments, wherein said cell is heterozygous for the modified pre-miRNA.

In a further embodiment, the invention relates to the method of any one of the preceding embodiments, wherein one or more guide sequences are introduced together with said nuclease.

In a further embodiment, the invention relates to a plant cell, preferably a Solanaceae, corn, rice, canola, soybean or sunflower cell, more preferably a tomato plant cell obtained by the method of any one of the preceding embodiments.

In a further embodiment, the invention relates to the plant cell of the preceding embodiment, wherein said cell comprises any one of SEQ ID NOs: 1-5.

In a further embodiment, the invention relates to the plant cell of the preceding embodiment, wherein said cell comprises any one of SEQ ID NOs: 8-17.

In a further embodiment, the invention relates to a method of producing plant seeds, preferably Solanaceae, corn, rice, canola, soybean or sunflower seeds, more preferably tomato seeds, comprising crossing a plant comprising a plant cell obtained by the method of any one of the preceding embodiments with itself or with another plant of the same crop.

### BRIEF DESCRIPTION OF THE DRAWINGS

FIG. 1 shows a graphical representation of the modification of a native pre-miRNA by swapping the native miRNA core for an amiRNA core complementary to the new target gene.

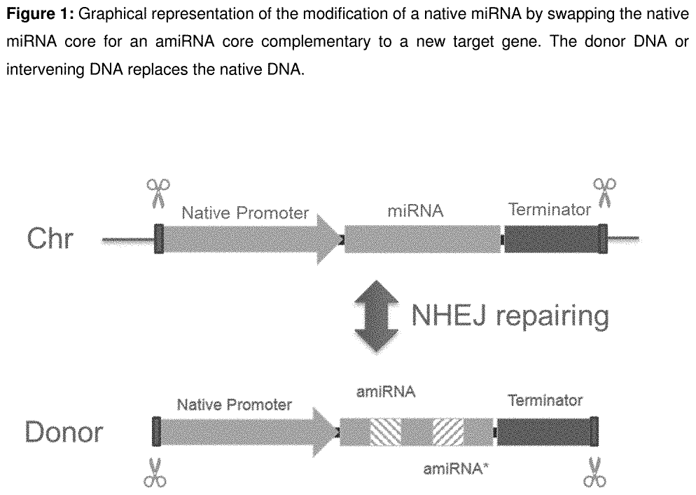

FIG. 2 shows the level of TSWV resistance in Nicotiana benthamiana plants with different overexpressed viral amiRNA core sequences.

FIG. 3 shows pictures of TSWV-infiltrated Nicotiana benthamiana plants with different overexpressed viral amiRNA core sequences.

FIG. 4 shows the level of TSWV resistance in Nicotiana benthamiana plants with different native pre-miRNA sequences modified with the viral amiRNA core of SEQ ID NO:

FIG. 5 shows binary vector 17839 (SEQ ID NO: 18) for transient experiments in Nicotiana benthamiana plants.

FIG. 6 shows binary vector 24598 (SEQ ID NO: 19) for tomato transformation with a soybean codon optimized Cas9 driven by constitutive prAtEF1aA1-02 promoter and two gene specific gRNAs driven by prAtU6-01 and prSIU6 to mutate tomato SlmiR156b gene (SEQ ID NO: 6).

### BRIEF DESCRIPTION OF THE SEQUENCES IN THE SEQUENCE LISTING

SEQ ID NO: 1 is the TSWV sequence of amiTSWV_N1w_PC (used as an amiRNA core in the context of the present invention)

SEQ ID NO: 2 is the TSWV sequence of amiTSWV_N2_PC (used as an amiRNA core in the context of the present invention)

SEQ ID NO: 3 is the TSWV sequence of amiTSWV_N2_PC_rev (used as an amiRNA core in the context of the present invention)

SEQ ID NO: 4 is the TSWV sequence of amiR159a_3p_N_GC35 (used as an amiRNA core in the context of the present invention)

SEQ ID NO: 5 is the TSWV sequence of amiR159a_3p_N_GC50 (used as an amiRNA core in the context of the present invention)

SEQ ID NO: 6 is the tomato sequence of miR156b,1 kb promoter included (used as a pre-miRNA scaffold in the context of the present invention)

SEQ ID NO: 7 is the tomato sequence of miR1919b, 1 kb promoter included (used as a pre-miRNA scaffold in the context of the present invention)

SEQ ID NOs: 8-12 are SEQ ID NOs: 1, 2, 3, 4 or 5 respectively embedded within SEQ ID NO: 6

SEQ ID NOs: 13-17 are SEQ ID NOs: 1, 2, 3, 4 or 5 respectively embedded within SEQ ID NO: 7

SEQ ID NO: 18 is the nucleotide sequence of binary vector 17839

SEQ ID NO: 19 is the nucleotide sequence of binary vector 24598.

SEQ ID NO: 20 and 21 are gRNA sequences.

SEQ ID NO: 22 is the TSWV sequence of amiTSWV_N1w_PC_rev (used as an amiRNA core in the context of the present invention)

SEQ ID NO: 23 is the TSWV sequence of amiR159a_3p_N_GC35_rev (used as an amiRNA core in the context of the present invention)

SEQ ID NO: 24 is the TSWV sequence of amiR159a_3p_N_GC50 (used as an amiRNA core in the context of the present invention)

### DETAILED DESCRIPTION OF THE INVENTION

This description is not intended to be a detailed catalogue of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure, which do not depart from the instant invention. Hence, the following descriptions are intended to illustrate some particular embodiments of the invention, and not to exhaustively specify all permutations, combinations and variations thereof.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention. All publications, patent applications, patents, and other references mentioned herein are incorporated by reference in their entirety.

The following definitions and methods are provided to better define the present invention and to guide those of ordinary skill in the art in the practice of the present invention. Unless otherwise noted, terms used herein are to be understood according to conventional usage by those of ordinary skill in the relevant art. Definitions of common terms in molecular biology may also be found in Rieger et al., Glossary of Genetics: Classical and Molecular, 5^{th} edition, Springer-Verlag, New York, 1994.

As used in the description of the embodiments of the invention and the appended claims, the singular forms “a,” “an,” and “the” are intended to include the plural forms as well, unless the context clearly indicates otherwise.

As used herein, “and/or” refers to and encompasses any and all possible combinations of one or more of the associated listed items.

The term “about,” as used herein when referring to a measurable value such as an amount of a compound, dose, time, temperature, and the like, is meant to encompass variations of 20%, 10%, 5%, 1%, 0.5%, or even 0.1% of the specified amount.

The terms “comprise,” “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the transitional phrase “consisting essentially of” means that the scope of a claim is to be interpreted to encompass the specified materials or steps recited in the claim and those that do not materially affect the basic and novel characteristic(s) of the claimed invention. Thus, the term “consisting essentially of” when used in a claim of this invention is not intended to be interpreted to be equivalent to “comprising.”

As used herein, the term “amplified” means the construction of multiple copies of a nucleic acid molecule or multiple copies complementary to the nucleic acid molecule using at least one of the nucleic acid molecules as a template. See, e.g., Diagnostic Molecular Microbiology: Principles and Applications, D. H. Persing et al., Ed., American Society for Microbiology, Washington, D.C. (1993). The product of amplification is termed an amplicon.

A “coding sequence” is a nucleic acid sequence that is transcribed into RNA such as mRNA, rRNA, tRNA, snRNA, sense RNA or antisense RNA. In some embodiments, the RNA is then translated in an organism to produce a protein.

As used herein the term transgenic “event” refers to a recombinant plant produced by transformation and regeneration of a single plant cell with heterologous DNA, for example, an expression cassette that includes one or more genes of interest (e.g., transgenes). The term “event” refers to the original transformant and/or progeny of the transformant that include the heterologous DNA. The term “event” also refers to progeny produced by a sexual outcross between the transformant and another line. Even after repeated backcrossing to a recurrent parent, the inserted DNA and the flanking DNA from the transformed parent is present in the progeny of the cross at the same chromosomal location. Normally, transformation of plant tissue produces multiple events, each of which represent insertion of a DNA construct into a different location in the genome of a plant cell. Based on the expression of the transgene or other desirable characteristics, a particular event is selected. Thus, “event MIR604,” “MIR604” or “MIR604 event” as used herein, means the original MIR604 transformant and/or progeny of the MIR604 transformant (U.S. Pat. Nos. 7,361,813, 7,897,748, 8,354,519, and 8,884,102, incorporated by references herein).

“Expression cassette” as used herein means a nucleic acid molecule capable of directing expression of a particular nucleotide sequence in an appropriate host cell, comprising a promoter operably linked to the nucleotide sequence of interest, typically a coding region, which is operably linked to termination signals. It also typically comprises sequences required for proper translation of the nucleotide sequence. The coding region usually codes for a protein of interest but may also code for a functional RNA of interest, for example antisense RNA or a nontranslated RNA, in the sense or antisense direction. The expression cassette may also comprise sequences not necessary in the direct expression of a nucleotide sequence of interest but which are present due to convenient restriction sites for removal of the cassette from an expression vector. The expression cassette comprising the nucleotide sequence of interest may be chimeric, meaning that at least one of its components is heterologous with respect to at least one of its other components. The expression cassette may also be one that is naturally occurring but has been obtained in a recombinant form useful for heterologous expression. Typically, however, the expression cassette is heterologous with respect to the host, i.e., the particular nucleic acid sequence of the expression cassette does not occur naturally in the host cell and must have been introduced into the host cell or an ancestor of the host cell by a transformation process known in the art. The expression of the nucleotide sequence in the expression cassette may be under the control of a constitutive promoter or of an inducible promoter that initiates transcription only when the host cell is exposed to some particular external stimulus. In the case of a multicellular organism, such as a plant, the promoter can also be specific to a particular tissue, or organ, or stage of development. An expression cassette, or fragment thereof, can also be referred to as “inserted sequence” or “insertion sequence” when transformed into a plant.

A “gene” is a defined region that is located within a genome and that, besides the aforementioned coding nucleic acid sequence, comprises other, primarily regulatory, nucleic acid sequences responsible for the control of the expression, that is to say the transcription and translation, of the coding portion. Genes can include both coding and non-coding regions (e.g., introns, regulatory elements, promoters, enhancers, termination sequences and 5′ and 3′ untranslated regions). A gene typically expresses mRNA, functional RNA, or specific protein, including regulatory sequences. Genes may or may not be capable of being used to produce a functional protein. In some embodiments, a gene refers to only the coding region. The term “native gene” refers to a gene as found in nature. The term “chimeric gene” refers to any gene that contains 1) DNA sequences, including regulatory and coding sequences that are not found together in nature, or 2) sequences encoding parts of proteins not naturally adjoined, or 3) parts of promoters that are not naturally adjoined. Accordingly, a chimeric gene may comprise regulatory sequences and coding sequences that are derived from different sources, or comprise regulatory sequences and coding sequences derived from the same source, but arranged in a manner different from that found in nature. A gene may be “isolated” by which is meant a nucleic acid molecule that is substantially or essentially free from components normally found in association with the nucleic acid molecule in its natural state. Such components include other cellular material, culture medium from recombinant production, and/or various chemicals used in chemically synthesizing the nucleic acid molecule.

By the term “express” or “expression” of a polynucleotide coding sequence, it is meant that the sequence is transcribed, and optionally translated.

A “gene of interest”, “nucleotide sequence of interest”, or “sequence of interest” refers to any gene which, when transferred to a plant, confers upon the plant a desired characteristic such as antibiotic resistance, virus resistance, insect resistance, disease resistance, or resistance to other pests, herbicide tolerance, improved nutritional value, improved performance in an industrial process or altered reproductive capability. The “gene of interest” may also be one that is transferred to plants for the production of commercially valuable enzymes or metabolites in the plant.

As used herein, “exogenous” refers to a nucleic acid molecule or nucleotide sequence not naturally associated with a host cell into which it is introduced, that either originates from another species or is from the same species or organism but is modified from either its original form or the form primarily expressed in the cell, including non-naturally occurring multiple copies of a naturally occurring nucleic acid sequence. Thus, a nucleotide sequence derived from an organism or species different from that of the cell into which the nucleotide sequence is introduced, is heterologous with respect to that cell and the cell's descendants. In addition, a heterologous nucleotide sequence includes a nucleotide sequence derived from and inserted into the same natural, original cell type, but which is present in a non-natural state, e.g., present in a different copy number, and/or under the control of different regulatory sequences than that found in the native state of the nucleic acid molecule. A nucleic acid sequence can also be heterologous to other nucleic acid sequences with which it may be associated, for example in a nucleic acid construct, such as e.g., an expression vector. As one non-limiting example, a promoter may be present in a nucleic acid construct in combination with one or more regulatory element and/or coding sequences that do not naturally occur in association with that particular promoter, i.e., they are heterologous to the promoter.

A “homologous” nucleic acid sequence is a nucleic acid sequence naturally associated with a host cell into which it is introduced. A homologous nucleic acid sequence can also be a nucleic acid sequence that is naturally associated with other nucleic acid sequences that may be present, e.g., in a nucleic acid construct. As one non-limiting example, a promoter may be present in a nucleic acid construct in combination with one or more regulatory elements and/or coding sequences that naturally occur in association with that particular promoter, i.e. they are homologous to the promoter.

“Operably-linked” refers to the association of nucleic acid sequences on a single nucleic acid sequence so that the function of one affects the function of the other. For example, a promoter is operably-linked with a coding sequence or functional RNA when it is capable of affecting the expression of that coding sequence or functional RNA (i.e. the coding sequence or functional RNA is under the transcriptional control of the promoter). Coding sequences in sense or antisense orientation can be operably-linked to regulatory sequences. Thus, regulatory or control sequences (e.g., promoters) operatively associated with a nucleotide sequence are capable of effecting expression of the nucleotide sequence. For example, a promoter operably linked to a nucleotide sequence encoding GFP would be capable of effecting the expression of that GFP nucleotide sequence.

The control sequences need not be contiguous with the nucleotide sequence of interest, as long as they function to direct the expression thereof. Thus, for example, intervening untranslated, yet transcribed, sequences can be present between a promoter and a coding sequence, and the promoter sequence can still be considered “operably linked” to the coding sequence.

“Primers” as used herein are isolated nucleic acids that are annealed to a complementary target DNA strand by nucleic acid hybridization to form a hybrid between the primer and the target DNA strand, then extended along the target DNA strand by a polymerase, such as DNA polymerase. Primer pairs or sets can be used for amplification of a nucleic acid molecule, for example, by the polymerase chain reaction (PCR) or other nucleic-acid amplification methods.

A “probe” is an isolated nucleic acid molecule that is complementary to a portion of a target nucleic acid molecule and is typically used to detect and/or quantify the target nucleic acid molecule. Thus, in some embodiments, a probe can be an isolated nucleic acid molecule to which is attached a detectable moiety or reporter molecule, such as a radioactive isotope, ligand, chemiluminescence agent, fluorescence agent or enzyme. Probes according to the present invention can include not only deoxyribonucleic or ribonucleic acids but also polyamides and other probe materials that bind specifically to a target nucleic acid sequence and can be used to detect the presence of and/or quantify the amount of, that target nucleic acid sequence.

A TaqMan probe is designed such that it anneals within a DNA region amplified by a specific set of primers. As the Taq polymerase extends the primer and synthesizes the nascent strand from a single-strand template from 3′ to 5′ of the complementary strand, the 5′ to 3′ exonuclease of the polymerase extends the nascent strand through the probe and consequently degrades the probe that has annealed to the template. Degradation of the probe releases the fluorophore from it and breaks the close proximity to the quencher, thus relieving the quenching effect and allowing fluorescence of the fluorophore. Hence, fluorescence detected in the quantitative PCR thermal cycler is directly proportional to the fluorophore released and the amount of DNA template present in the PCR.

Primers and probes are generally between 5 and 100 nucleotides or more in length. In some embodiments, primers and probes can be at least 20 nucleotides or more in length, or at least 25 nucleotides or more, or at least 30 nucleotides or more in length. Such primers and probes hybridize specifically to a target sequence under optimum hybridization conditions as are known in the art. Primers and probes according to the present invention may have complete sequence complementarity with the target sequence, although probes differing from the target sequence and which retain the ability to hybridize to target sequences may be designed by conventional methods according to the invention.

Methods for preparing and using probes and primers are described, for example, in Molecular Cloning: A Laboratory Manual, 2nd ed., vol. 1-3, ed. Sambrook et al., Cold Spring Harbor Laboratory Press, Cold Spring Harbor, N.Y., 1989. PCR-primer pairs can be derived from a known sequence, for example, by using computer programs intended for that purpose.

The polymerase chain reaction (PCR) is a technique for “amplifying” a particular piece of DNA. In order to perform PCR, at least a portion of the nucleotide sequence of the DNA molecule to be replicated must be known. In general, primers or short oligonucleotides are used that are complementary (e.g., substantially complementary or fully complementary) to the nucleotide sequence at the 3′ end of each strand of the DNA to be amplified (known sequence). The DNA sample is heated to separate its strands and is mixed with the primers. The primers hybridize to their complementary sequences in the DNA sample. Synthesis begins (5′ to 3′ direction) using the original DNA strand as the template. The reaction mixture must contain all four deoxynucleotide triphosphates (dATP, dCTP, dGTP and dTTP) and a DNA polymerase. Polymerization continues until each newly-synthesized strand has proceeded far enough to contain the sequence recognized by the other primer. Once this occurs, two DNA molecules are created that are identical to the original molecule. These two molecules are heated to separate their strands and the process is repeated. Each cycle doubles the number of DNA molecules. Using automated equipment, each cycle of replication can be completed in less than 5 minutes. After 30 cycles, what began as a single molecule of DNA has been amplified into more than a billion copies (2³⁰=1.02×10⁹).

The oligonucleotides of an oligonucleotide primer pair are complementary to DNA sequences located on opposite DNA strands and flanking the region to be amplified. The annealed primers hybridize to the newly synthesized DNA strands. The first amplification cycle will result in two new DNA strands whose 5′ end is fixed by the position of the oligonucleotide primer but whose 3′ end is variable (‘ragged’ 3′ ends). The two new strands can serve in turn as templates for synthesis of complementary strands of the desired length (the 5′ ends are defined by the primer and the 3′ ends are fixed because synthesis cannot proceed past the terminus of the opposing primer). After a few cycles, the desired fixed length product begins to predominate.

A quantitative polymerase chain reaction (qPCR), also referred to as real-time polymerase chain reaction, monitors the accumulation of a DNA product from a PCR reaction in real time. qPCR is a laboratory technique of molecular biology based on the polymerase chain reaction (PCR), which is used to amplify and simultaneously quantify a targeted DNA molecule. Even one copy of a specific sequence can be amplified and detected in PCR. The PCR reaction generates copies of a DNA template exponentially. This results in a quantitative relationship between the amount of starting target sequence and amount of PCR product accumulated at any particular cycle. Due to inhibitors of the polymerase reaction found with the template, reagent limitation or accumulation of pyrophosphate molecules, the PCR reaction eventually ceases to generate template at an exponential rate (i.e., the plateau phase), making the end point quantitation of PCR products unreliable. Therefore, duplicate reactions may generate variable amounts of PCR product. Only during the exponential phase of the PCR reaction is it possible to extrapolate back in order to determine the starting quantity of template sequence. The measurement of PCR products as they accumulate (i.e., real-time quantitative PCR) allows quantitation in the exponential phase of the reaction and therefore removes the variability associated with conventional PCR. In a real time PCR assay, a positive reaction is detected by accumulation of a fluorescent signal. For one or more specific sequences in a DNA sample, quantitative PCR enables both detection and quantification. The quantity can be either an absolute number of copies or a relative amount when normalized to DNA input or additional normalizing genes. Since the first documentation of real-time PCR, it has been used for an increasing and diverse number of applications including mRNA expression studies, DNA copy number measurements in genomic or viral DNAs, allelic discrimination assays, expression analysis of specific splice variants of genes and gene expression in paraffin-embedded tissues and laser captured micro-dissected cells.

As used herein, the phrase “Ct value” refers to “threshold cycle,” which is defined as the “fractional cycle number at which the amount of amplified target reaches a fixed threshold.” In some embodiments, it represents an intersection between an amplification curve and a threshold line. The amplification curve is typically in an “S” shape indicating the change of relative fluorescence of each reaction (Y-axis) at a given cycle (X-axis), which in some embodiments is recorded during PCR by a real-time PCR instrument. The threshold line is in some embodiments the level of detection at which a reaction reaches a fluorescence intensity above background. See Livak & Schmittgen (2001) 25 Methods 402-408. It is a relative measure of the concentration of the target in the PCR. Generally, good Ct values for quantitative assays such as qPCR are in some embodiments in the range of 10-40 for a given reference gene. Ct levels are inversely proportional to the amount of target nucleic acid in the sample (i.e. the lower the Ct level the greater the amount of detectable target nucleic acid in the sample). Additionally, good Ct values for quantitative assays such as qPCR show a linear response range with proportional dilutions of target gDNA.

In some embodiments, qPCR is performed under conditions wherein the Ct value can be collected in real-time for quantitative analysis. For example, in a typical qPCR experiment, DNA amplification is monitored at each cycle of PCR during the extension stage. The amount of fluorescence generally increases above the background when DNA is in the log linear phase of amplification. In some embodiments, the Ct value is collected at this time point.

As used herein, the term “cell” refers to any living cell. The cell may be a prokaryotic or eukaryotic cell. The cell may be isolated. The cell may or may not be capable of regenerating into an organism. The cell may be in the context of a tissue, callus, culture, organ, or part. In some embodiments, the cell may be a plant cell. A plant cell of the present invention can be in the form of an isolated single cell or can be a cultured cell or can be a part of a higher-organized unit such as, for example, a plant tissue or a plant organ. The plant cell may be derived from or part of an angiosperm or gymnosperm. In further embodiments, the plant cell may be a monocotyledonous plant cell, a dicotyledonous plant cell. The monocotyledonous plant cell may be, for example, a maize, rice, sorghum, sugarcane, barley, wheat, oat, turf grass, or ornamental grass cell. The dicotyledonous plant cell may be, for example, a tobacco, pepper, eggplant, sunflower, crucifer, flax, potato, cotton, soybean, sugar bee, or oilseed rape cell.

The term “plant part,” as used herein, includes but is not limited to embryos, pollen, ovules, seeds, leaves, stems, shoots, flowers, branches, fruit, kernels, ears, cobs, husks, stalks, roots, root tips, anthers, plant cells including plant cells that are intact in plants and/or parts of plants, plant protoplasts, plant tissues, plant cell tissue cultures, plant calli, plant clumps, and the like. As used herein, “shoot” refers to the above ground parts including the leaves and stems. Further, as used herein, “plant cell” refers to a structural and physiological unit of the plant, which comprises a cell wall and also may refer to a protoplast.

The term “introducing” or “introduce” in the context of a cell, prokaryotic cell, bacterial cell, eukaryotic cell, plant cell, plant and/or plant part means contacting a nucleic acid molecule with the cell, eukaryotic cell, plant, plant part, and/or plant cell in such a manner that the nucleic acid molecule gains access to the interior of the cell, eukaryotic cell, plant cell and/or a cell of the plant and/or plant part. Where more than one nucleic acid molecule is to be introduced these nucleic acid molecules can be assembled as part of a single polynucleotide or nucleic acid construct, or as separate polynucleotide or nucleic acid constructs, and can be located on the same or different nucleic acid constructs. Accordingly, these polynucleotides can be introduced into plant cells in a single transformation event, in separate transformation events, or, e.g., as part of a breeding protocol through conventional crossing.

An “inversion” is a chromosome rearrangement in which a segment of a chromosome is reversed end to end. An inversion occurs when a single chromosome undergoes breakage and rearrangement within itself. A chromosome “translocation” is a rearrangement of parts between non-homologous chromosomes.

As used herein, the terms “transformed” and “transgenic” refer to any cell, prokaryotic cell, eukaryotic cell, plant, plant cell, callus, plant tissue, or plant part that contains all or part of at least one recombinant (e.g., heterologous) polynucleotide. In some embodiments, all or part of the recombinant polynucleotide is stably integrated into a chromosome or stable extra-chromosomal element, so that it is passed on to successive generations. For the purposes of the invention, the term “recombinant polynucleotide” refers to a polynucleotide that has been altered, rearranged, or modified by genetic engineering. Examples include any cloned polynucleotide, or polynucleotides, that are linked or joined to heterologous sequences. The term “recombinant” does not refer to alterations of polynucleotides that result from naturally occurring events, such as spontaneous mutations, or from non-spontaneous mutagenesis followed by selective breeding.

The term “transformation” as used herein refers to the introduction of a heterologous nucleic acid into a cell. Transformation of a cell may be stable or transient. Thus, a transgenic cell, plant cell, plant and/or plant part of the invention can be stably transformed or transiently transformed. Transformation can refer to the transfer of a nucleic acid molecule into the genome of a host cell, resulting in genetically stable inheritance. In some embodiments, the introduction into a plant, plant part and/or plant cell is via bacterial-mediated transformation, particle bombardment transformation, calcium-phosphate-mediated transformation, cyclodextrin-mediated transformation, electroporation, liposome-mediated transformation, nanoparticle-mediated transformation, polymer-mediated transformation, virus-mediated nucleic acid delivery, whisker-mediated nucleic acid delivery, microinjection, sonication, infiltration, polyethylene glycol-mediated transformation, protoplast transformation, or any other electrical, chemical, physical and/or biological mechanism that results in the introduction of nucleic acid into the plant, plant part and/or cell thereof, or any combination thereof.

Procedures for transforming plants are well known and routine in the art and are described throughout the literature. Non-limiting examples of methods for transformation of plants include transformation via bacterial-mediated nucleic acid delivery (e.g. via bacteria from the genus Agrobacterium), viral-mediated nucleic acid delivery, silicon carbide or nucleic acid whisker-mediated nucleic acid delivery, liposome mediated nucleic acid delivery, microinjection, microparticle bombardment, calcium-phosphate-mediated transformation, cyclodextrin-mediated transformation, electroporation, nanoparticle-mediated transformation, sonication, infiltration, PEG-mediated nucleic acid uptake, as well as any other electrical, chemical, physical (mechanical) and/or biological mechanism that results in the introduction of nucleic acid into the plant cell, including any combination thereof. General guides to various plant transformation methods known in the art include Miki et al. (“Procedures for Introducing Foreign DNA into Plants” in Methods in Plant Molecular Biology and Biotechnology, Glick, B. R. and Thompson, J. E., Eds. (CRC Press, Inc., Boca Raton, 1993), pages 67-88) and Rakowoczy-Trojanowska (Cell Mol Biol Lett 7:849-858 (2002)).

Agrobacterium-mediated transformation is a commonly used method for transforming plants because of its high efficiency of transformation and because of its broad utility with many different species. Agrobacterium-mediated transformation typically involves transfer of the binary vector carrying the foreign DNA of interest to an appropriate Agrobacterium strain that may depend on the complement of vir genes carried by the host Agrobacterium strain either on a co-resident Ti plasmid or chromosomally (Uknes et al. 1993, Plant Cell 5:159-169). The transfer of the recombinant binary vector to Agrobacterium can be accomplished by a tri-parental mating procedure using Escherichia coli carrying the recombinant binary vector, a helper E. coli strain that carries a plasmid that is able to mobilize the recombinant binary vector to the target Agrobacterium strain. Alternatively, the recombinant binary vector can be transferred to Agrobacterium by nucleic acid transformation (Hofgen and Willmitzer 1988, Nucleic Acids Res 16:9877).

Transformation of a plant by recombinant Agrobacterium usually involves co-cultivation of the Agrobacterium with explants from the plant and follows methods well known in the art. Transformed tissue is typically regenerated on selection medium carrying an antibiotic or herbicide resistance marker between the binary plasmid T-DNA borders. An exemplary method for transforming tomato plants is disclosed in Garcia D., Narváez-Vásquez J., Orozco-Cárdenas M. L. (2015) Tomato (Solanum lycopersicum). In: Wang K. (eds) Agrobacterium Protocols. Methods in Molecular Biology, vol 1223. Springer, New York, N.Y.

Another method for transforming plants, plant parts and plant cells involves propelling inert or biologically active particles at plant tissues and cells. See, e.g., US Pat. Nos. 4,945,050; 5,036,006 and 5,100,792. Generally, this method involves propelling inert or biologically active particles at the plant cells under conditions effective to penetrate the outer surface of the cell and afford incorporation within the interior thereof. When inert particles are utilized, the vector can be introduced into the cell by coating the particles with the vector containing the nucleic acid of interest. Alternatively, a cell or cells can be surrounded by the vector so that the vector is carried into the cell by the wake of the particle. Biologically active particles (e.g., dried yeast cells, dried bacteria or a bacteriophage, each containing one or more nucleic acids sought to be introduced) also can be propelled into plant tissue.

“Transient transformation” in the context of a polynucleotide means that a polynucleotide is introduced into the cell and does not integrate into the genome of the cell.

As used herein, “stably introducing,” “stably introduced,” “stable transformation” or “stably transformed” in the context of a polynucleotide introduced into a cell, means that the introduced polynucleotide is stably integrated into the genome of the cell, and thus the cell is stably transformed with the polynucleotide. As such, the integrated polynucleotide is capable of being inherited by the progeny thereof, more particularly, by the progeny of multiple successive generations. “Genome” as used herein includes the nuclear and/or plastid genome, and therefore includes integration of a polynucleotide into, for example, the chloroplast genome. Stable transformation as used herein can also refer to a polynucleotide that is maintained extrachromasomally, for example, as a minichromosome.

Transient transformation may be detected by, for example, an enzyme-linked immunosorbent assay (ELISA) or Western blot, which can detect the presence of a peptide or polypeptide encoded by one or more nucleic acid molecules introduced into an organism. Stable transformation of a cell can be detected by, for example, a Southern blot hybridization assay of genomic DNA of the cell with nucleic acid sequences which specifically hybridize with a nucleotide sequence of a nucleic acid molecule introduced into an organism (e.g., a plant). Stable transformation of a cell can be detected by, for example, a Northern blot hybridization assay of RNA of the cell with nucleic acid sequences which specifically hybridize with a nucleotide sequence of a nucleic acid molecule introduced into a plant or other organism. Stable transformation of a cell can also be detected by, e.g., a polymerase chain reaction (PCR) or other amplification reaction as are well known in the art, employing specific primer sequences that hybridize with target sequence(s) of a nucleic acid molecule, resulting in amplification of the target sequence(s), which can be detected according to standard methods. Transformation can also be detected by direct sequencing and/or hybridization protocols well known in the art.

Thus, in particular embodiments of the present invention, a plant cell can be transformed by any method known in the art and as described herein and intact plants can be regenerated from these transformed cells using any of a variety of known techniques. Plant regeneration from plant cells, plant tissue culture and/or cultured protoplasts is described, for example, in Evans et al. (Handbook of Plant Cell Cultures, Vol. 1, MacMilan Publishing Co. New York (1983)); and Vasil I. R. (ed.) (Cell Culture and Somatic Cell Genetics of Plants, Acad. Press, Orlando, Vol. I (1984), and Vol. II (1986)). Methods of selecting for transformed transgenic plants, plant cells and/or plant tissue culture are routine in the art and can be employed in the methods of the invention provided herein.

The “transformation and regeneration process” refers to the process of stably introducing a transgene into a plant cell and regenerating a plant from the transgenic plant cell. As used herein, transformation and regeneration includes the selection process, whereby a transgene comprises a selectable marker and the transformed cell has incorporated and expressed the transgene, such that the transformed cell will survive and developmentally flourish in the presence of the selection agent. “Regeneration” refers to growing a whole plant from a plant cell, a group of plant cells, or a plant piece such as from a protoplast, callus, or tissue part.

The terms “nucleotide sequence” “nucleic acid,” “nucleic acid sequence,” “nucleic acid molecule,” “oligonucleotide” and “polynucleotide” are used interchangeably herein to refer to a heteropolymer of nucleotides and encompass both RNA and DNA, including cDNA, genomic DNA, mRNA, synthetic (e.g., chemically synthesized) DNA or RNA and chimeras of RNA and DNA. The term nucleic acid molecule refers to a chain of nucleotides without regard to length of the chain. The nucleotides contain a sugar, phosphate and a base which is either a purine or pyrimidine. A nucleic acid molecule can be double-stranded or single-stranded. Where single-stranded, the nucleic acid molecule can be a sense strand or an antisense strand. A nucleic acid molecule can be synthesized using oligonucleotide analogs or derivatives (e.g., inosine or phosphorothioate nucleotides). Such oligonucleotides can be used, for example, to prepare nucleic acid molecules that have altered base-pairing abilities or increased resistance to nucleases. Nucleic acid sequences provided herein are presented herein in the 5′ to 3′ direction, from left to right and are represented using the standard code for representing the nucleotide characters as set forth in the U.S. sequence rules, 37 CFR §§ 1.821-1.825 and the World Intellectual Property Organization (WIPO) Standard ST.25.

A “nucleic acid fragment” is a fraction of a given nucleic acid molecule. An “RNA fragment” is a fraction of a given RNA molecule. A “DNA fragment” is a fraction of a given DNA molecule. A “nucleic acid segment” is a fraction of a given nucleic acid molecule and is not isolated from the molecule. An “RNA segment” is a fraction of a given RNA molecule and is not isolated from the molecule. A “DNA segment” is a fraction of a given DNA molecule and is not isolated from the molecule. Segments of polynucleotides can be any length, for example, at least 5, 10, 15, 20, 25, 30, 40, 50, 7 5, 100, 150, 200, 300 or 500 or more nucleotides in length. A segment or portion of a guide sequence can be about 50%, 40%, 30%, 20%, 10% of the guide sequence, e.g., one-third of the guide sequence or shorter, e.g., 7, 6,5,4,3, or 2 nucleotides in length.

The term “derived from” in the context of a molecule refers to a molecule isolated or made using a parent molecule or information from that parent molecule. For example, a Cas9 single mutant nickase and a Cas9 double mutant null-nuclease are derived from a wild-type Cas9 protein.

In higher plants, deoxyribonucleic acid (DNA) is the genetic material while ribonucleic acid (RNA) is involved in the transfer of information contained within DNA into proteins. A “genome” is the entire body of genetic material contained in each cell of an organism. Unless otherwise indicated, a particular nucleic acid sequence of this invention also implicitly encompasses conservatively modified variants thereof (e.g., degenerate codon substitutions) and complementary sequences and as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). The term nucleic acid molecule is used interchangeably with gene, cDNA, and mRNA encoded by a gene.

As used herein “sequence identity” refers to the extent to which two optimally aligned polynucleotide or peptide sequences are invariant throughout a window of alignment of components, e.g., nucleotides or amino acids. “Identity” can be readily calculated by known methods including, but not limited to, those described in: Computational Molecular Biology (Lesk, A. M., ed.) Oxford University Press, New York (1988); Biocomputing: Informatics and Genome Projects (Smith, D. W., ed.) Academic Press, New York (1993); Computer Analysis of Sequence Data, Part I (Griffin, A. M., and Griffin, H. G., eds.) Humana Press, New Jersey (1994); Sequence Analysis in Molecular Biology (von Heinje, G., ed.) Academic Press (1987); and Sequence Analysis Primer (Gribskov, M. and Devereux, J., eds.) Stockton Press, New York (1991).

As used herein, the term “percent sequence identity” or “percent identity” refers to the percentage of identical nucleotides in a linear polynucleotide sequence of a reference (“query”) polynucleotide molecule (or its complementary strand) as compared to a test (“subject”) polynucleotide molecule (or its complementary strand) when the two sequences are optimally aligned. In some embodiments, “percent identity” can refer to the percentage of identical amino acids in an amino acid sequence.

As used herein, the phrase “substantially identical,” in the context of two nucleic acid molecules, nucleotide sequences or protein sequences, refers to two or more sequences or subsequences that have at least about 70%, least about 75%, at least about 80%, least about 85%, at least about 90%, at least about 95%, at least about 96%, at least about 97%, at least about 98%, or at least about 99% nucleotide or amino acid residue identity, when compared and aligned for maximum correspondence, as measured using one of the following sequence comparison algorithms or by visual inspection. In some embodiments of the invention, the substantial identity exists over a region of the sequences that is at least about 50 residues to about 150 residues in length. Thus, in some embodiments of this invention, the substantial identity exists over a region of the sequences that is at least about 50, about 60, about 70, about 80, about 90, about 100, about 110, about 120, about 130, about 140, about 150, or more residues in length. In some particular embodiments, the sequences are substantially identical over at least about 150 residues. In a further embodiment, the sequences are substantially identical over the entire length of the coding regions. Furthermore, in representative embodiments, substantially identical nucleotide or protein sequences perform substantially the same function (e.g., guiding to a particular genomic target, endonuclease cleavage of a particular genomic target site).

For sequence comparison, typically one sequence acts as a reference sequence to which test sequences are compared. When using a sequence comparison algorithm, test and reference sequences are entered into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and optionally by computerized implementations of these algorithms such as GAP, BESTFIT, FASTA, and TFASTA available as part of the GCG® Wisconsin Package® (Accelrys Inc., San Diego, Calif.). An “identity fraction” for aligned segments of a test sequence and a reference sequence is the number of identical components which are shared by the two aligned sequences divided by the total number of components in the reference sequence segment, i.e., the entire reference sequence or a smaller defined part of the reference sequence. Percent sequence identity is represented as the identity fraction multiplied by 100. The comparison of one or more polynucleotide sequences may be to a full-length polynucleotide sequence or a portion thereof, or to a longer polynucleotide sequence. For purposes of this invention “percent identity” may also be determined using BLASTX version 2.0 for translated nucleotide sequences and BLASTN version 2.0 for polynucleotide sequences.

Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighbourhood word score threshold (Altschul et al., 1990). These initial neighbourhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89: 10915 (1989)).

In addition to calculating percent sequence identity, the BLAST algorithm also performs a statistical analysis of the similarity between two sequences (see, e.g., Karlin & Altschul, Proc. Natl. Acad. Sci. USA 90: 5873-5787 (1993)). One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleotide sequence to the reference nucleotide sequence is less than about 0.1 to less than about 0.001. Thus, in some embodiments of the invention, the smallest sum probability in a comparison of the test nucleotide sequence to the reference nucleotide sequence is less than about 0.001.

Two nucleotide sequences can also be considered to be substantially identical when the two sequences hybridize to each other under stringent conditions. In some representative embodiments, two nucleotide sequences considered to be substantially identical hybridize to each other under highly stringent conditions.

“Stringent hybridization conditions” and “stringent hybridization wash conditions” in the context of nucleic acid hybridization experiments such as Southern and Northern hybridizations are sequence dependent, and are different under different environmental parameters. An extensive guide to the hybridization of nucleic acids is found in Tijssen Laboratory Techniques in Biochemistry and Molecular Biology-Hybridization with Nucleic Acid Probes part I chapter 2 “Overview of principles of hybridization and the strategy of nucleic acid probe assays” Elsevier, New York (1993). Generally, highly stringent hybridization and wash conditions are selected to be about 5° C. lower than the thermal melting point (Tₘ) for the specific sequence at a defined ionic strength and pH.

The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for hybridization of complementary nucleotide sequences which have more than 100 complementary residues on a filter in a Southern or northern blot is 50% formamide with 1 mg of heparin at 42° C., with the hybridization being carried out overnight. An example of highly stringent wash conditions is 0.1 5M NaCl at 72° C. for about 15 minutes. An example of stringent wash conditions is a 0.2×SSC wash at 65° C. for 15 minutes (see, Sambrook, infra, for a description of SSC buffer). Often, a high stringency wash is preceded by a low stringency wash to remove background probe signal. An example of a medium stringency wash for a duplex of, e.g., more than 100 nucleotides, is 1×SSC at 45° C. for 15 minutes. An example of a low stringency wash for a duplex of, e.g., more than 100 nucleotides, is 4-6×SSC at 40° C. for 15 minutes. For short probes (e.g., about 10 to 50 nucleotides), stringent conditions typically involve salt concentrations of less than about 1.0 M Na ion, typically about 0.01 to 1.0 M Na ion concentration (or other salts) at pH 7.0 to 8.3, and the temperature is typically at least about 30° C. Stringent conditions can also be achieved with the addition of destabilizing agents such as formamide. In general, a signal to noise ratio of 2× (or higher) than that observed for an unrelated probe in the particular hybridization assay indicates detection of a specific hybridization. Nucleotide sequences that do not hybridize to each other under stringent conditions are still substantially identical if the proteins that they encode are substantially identical. This can occur, for example, when a copy of a nucleotide sequence is created using the maximum codon degeneracy permitted by the genetic code.

The following are examples of sets of hybridization/wash conditions that may be used to clone homologous nucleotide sequences that are substantially identical to reference nucleotide sequences of the present invention. In one embodiment, a reference nucleotide sequence hybridizes to the “test” nucleotide sequence in 7% sodium dodecyl sulfate (SDS), 0.5 M NaPO₄, 1 mM EDTA at 50° C. with washing in 2×SSC, 0.1% SDS at 50° C. In another embodiment, the reference nucleotide sequence hybridizes to the “test” nucleotide sequence in 7% sodium dodecyl sulfate (SDS), 0.5 M NaPO₄, 1 mM EDTA at 50° C. with washing in 1×SSC, 0.1% SDS at 50° C. or in 7% sodium dodecyl sulfate (SDS), 0.5 M NaPO₄, 1 mM EDTA at 50° C. with washing in 0.5×SSC, 0.1% SDS at 50° C. In still further embodiments, the reference nucleotide sequence hybridizes to the “test” nucleotide sequence in 7% sodium dodecyl sulfate (SDS), 0.5 M NaPO₄, 1 mM EDTA at 50° C. with washing in 0.1×SSC, 0.1% SDS at 50° C., or in 7% sodium dodecyl sulfate (SDS), 0.5 M NaPO₄, 1 mM EDTA at 50° C. with washing in 0.1×SSC, 0.1% SDS at 65° C.

An “isolated” nucleic acid molecule or nucleotide sequence or an “isolated” polypeptide is a nucleic acid molecule, nucleotide sequence or polypeptide that, by the hand of man, exists apart from its native environment and/or has a function that is different, modified, modulated and/or altered as compared to its function in its native environment and is therefore not a product of nature. An isolated nucleic acid molecule or isolated polypeptide may exist in a purified form or may exist in a non-native environment such as, for example, a recombinant host cell. Thus, for example, with respect to polynucleotides, the term isolated means that it is separated from the chromosome and/or cell in which it naturally occurs. A polynucleotide is also isolated if it is separated from the chromosome and/or cell in which it naturally occurs and is then inserted into a genetic context, a chromosome, a chromosome location, and/or a cell in which it does not naturally occur. The recombinant nucleic acid molecules and nucleotide sequences of the invention can be considered to be “isolated” as defined above.

Thus, an “isolated nucleic acid molecule” or “isolated nucleotide sequence” is a nucleic acid molecule or nucleotide sequence that is not immediately contiguous with nucleotide sequences with which it is immediately contiguous (one on the 5′ end and one on the 3′ end) in the naturally occurring genome of the organism from which it is derived. Accordingly, in one embodiment, an isolated nucleic acid includes some or all of the 5′ non-coding (e.g., promoter) sequences that are immediately contiguous to a coding sequence. The term therefore includes, for example, a recombinant nucleic acid that is incorporated into a vector, into an autonomously replicating plasmid or virus, or into the genomic DNA of a prokaryote or eukaryote, or which exists as a separate molecule (e.g., a cDNA or a genomic DNA fragment produced by PCR or restriction endonuclease treatment), independent of other sequences. It also includes a recombinant nucleic acid that is part of a hybrid nucleic acid molecule encoding an additional polypeptide or peptide sequence. An “isolated nucleic acid molecule” or “isolated nucleotide sequence” can also include a nucleotide sequence derived from and inserted into the same natural, original cell type, but which is present in a non-natural state, e.g., present in a different copy number, and/or under the control of different regulatory sequences than that found in the native state of the nucleic acid molecule.

The term “isolated” can further refer to a nucleic acid molecule, nucleotide sequence, polypeptide, peptide or fragment that is substantially free of cellular material, viral material, and/or culture medium (e.g., when produced by recombinant DNA techniques), or chemical precursors or other chemicals (e.g., when chemically synthesized). Moreover, an “isolated fragment” is a fragment of a nucleic acid molecule, nucleotide sequence or polypeptide that is not naturally occurring as a fragment and would not be found as such in the natural state. “Isolated” does not necessarily mean that the preparation is technically pure (homogeneous), but it is sufficiently pure to provide the polypeptide or nucleic acid in a form in which it can be used for the intended purpose.

In representative embodiments of the invention, an “isolated” nucleic acid molecule, nucleotide sequence, and/or polypeptide is at least about 5%, 10%, 15%, 20%, 25%, 30%, 40%, 50%, 60%, 70%, 75%, 80%, 85%, 90%, 95%, 97%, 98%, 99% pure (w/w) or more. In other embodiments, an “isolated” nucleic acid, nucleotide sequence, and/or polypeptide indicates that at least about a 5-fold, 10-fold, 25-fold, 100-fold, 1000-fold, 10,000-fold, 100,000-fold or more enrichment of the nucleic acid (w/w) is achieved as compared with the starting material.

“Wild-type” nucleotide sequence or amino acid sequence refers to a naturally occurring (“native ”) or endogenous nucleotide sequence or amino acid sequence. Thus, for example, a “wild-type mRNA” is an mRNA that is naturally occurring in or endogenous to the organism. A “homologous” nucleotide sequence is a nucleotide sequence naturally associated with a host cell into which it is introduced.

The terms “open reading frame” and “ORF” refer to the amino acid sequence encoded between translation initiation and termination codons of a coding sequence. The terms “initiation codon” and “termination codon” refer to a unit of three adjacent nucleotides (‘codon’) in a coding sequence that specifies initiation and chain termination, respectively, of protein synthesis (mRNA translation).

“Promoter” refers to a nucleotide sequence, usually upstream (5′) to its coding sequence, which controls the expression of the coding sequence by providing the recognition for RNA polymerase and other factors required for proper transcription. “Promoter regulatory sequences” consist of proximal and more distal upstream elements. Promoter regulatory sequences influence the transcription, RNA processing or stability, or translation of the associated coding sequence. Regulatory sequences include enhancers, promoters, untranslated leader sequences, introns, and polyadenylation signal sequences. They include natural and synthetic sequences as well as sequences that may be a combination of synthetic and natural sequences. An “enhancer” is a DNA sequence that can stimulate promoter activity and may be an innate element of the promoter or a heterologous element inserted to enhance the level or tissue specificity of a promoter. It is capable of operating in both orientations (normal or flipped), and is capable of functioning even when moved either upstream or downstream from the promoter. The meaning of the term “promoter” includes “promoter regulatory sequences.”

“Primary transformant” and “EO generation” refer to transgenic plants that are of the same genetic generation as the tissue that was initially transformed (i.e., not having gone through meiosis and fertilization since transformation). “Secondary transformants” and the “E1, E2, E3, etc. generations” refer to transgenic plants derived from primary transformants through one or more meiotic and fertilization cycles. They may be derived by self-fertilization of primary or secondary transformants or crosses of primary or secondary transformants with other transformed or untransformed plants.

A “transgene” refers to a nucleic acid molecule that has been introduced into the genome by transformation and is stably maintained. A transgene may comprise at least one expression cassette, typically comprises at least two expression cassettes, and may comprise ten or more expression cassettes. Transgenes may include, for example, genes that are either heterologous or homologous to the genes of a particular plant to be transformed. Additionally, transgenes may comprise native genes inserted into a non-native organism, or chimeric genes. The term “endogenous gene” refers to a native gene in its natural location in the genome of an organism. A “foreign” gene refers to a gene not normally found in the host organism but one that is introduced into the organism by gene transfer.

“Intron” refers to an intervening section of DNA which occurs almost exclusively within a eukaryotic gene, but which is not translated to amino acid sequences in the gene product. The introns are removed from the pre-mature mRNA through a process called splicing, which leaves the exons untouched, to form an mRNA. For purposes of the present invention, the definition of the term “intron” includes modifications to the nucleotide sequence of an intron derived from a target gene, provided the modified intron does not significantly reduce the activity of its associated 5′ regulatory sequence.

“Exon” refers to a section of DNA which carries the coding sequence for a protein or part of it. Exons are separated by intervening, non-coding sequences (introns). For purposes of the present invention, the definition of the term “exon” includes modifications to the nucleotide sequence of an exon derived from a target gene, provided the modified exon does not significantly reduce the activity of its associated 5′ regulatory sequence.

The term “cleavage” or “cleaving” refers to breaking of the covalent phosphodiester linkage in the ribosylphosphodiester backbone of a polynucleotide. The terms “cleavage” or “cleaving” encompass both single-stranded breaks and double-stranded breaks. Double-stranded cleavage can occur as a result of two distinct single-stranded cleavage events. Cleavage can result in the production of either blunt ends or staggered ends. A “nuclease cleavage site” or “genomic nuclease cleavage site” is a region of nucleotides that comprise a nuclease cleavage sequence that is recognized by a specific nuclease, which acts to cleave the nucleotide sequence of the genomic DNA in one or both strands. Such cleavage by the nuclease enzyme initiates DNA repair mechanisms within the cell, which establishes an environment for homologous recombination to occur.

A “donor molecule” or “donor sequence” is a nucleotide polymer or oligomer intended for insertion at a target polynucleotide, typically a target genomic site. The donor sequence may be one or more transgenes, expression cassettes, or nucleotide sequences of interest. A donor molecule may be a donor DNA molecule, either single stranded, partially double-stranded, or double-stranded. The donor polynucleotide may be a natural or a modified polynucleotide, a RNA-DNA chimera, or a DNA fragment, either single- or at least partially double-stranded, or a fully double-stranded DNA molecule, or a PGR amplified ssDNA or at least partially dsDNA fragment. In some embodiments, the donor DNA molecule is part of a circularized DNA molecule. A fully double-stranded donor DNA is advantageous since it might provide an increased stability, since dsDNA fragments are generally more resistant than ssDNA to nuclease degradation. In some embodiments, the donor polynucleotide molecule can comprise at least about 100, 150, 200, 250, 300, 250, 400, 450, 500, 600, 700, 800, 900, 1000, 1500, 2000, 2500, 3000, 3500, 4000, 4500, 5000, 7500, 10000, 15,000 or 20,000 nucleotides, including any value within this range not explicitly recited herein. In some embodiments, the donor DNA molecule comprises heterologous nucleic acid sequence. In some embodiments, the donor DNA molecule comprises at least one expression cassette. In some embodiments, the donor DNA molecule may comprise a transgene, which comprises at least one expression cassette. In some embodiments, the donor DNA molecule comprises an allelic modification of a gene which is native to the target genome. The allelic modification may comprise at least one nucleotide insertion, at least one nucleotide deletion, and/or at least one nucleotide substitution. In some embodiments, the allelic modification may comprise an INDEL. In some embodiments, the donor DNA molecule comprises homologous arms to the target genomic site. In some embodiments, the donor DNA molecule comprises at least 100 contiguous nucleotides at least 90% identical to a genomic nucleic acid sequence, and optionally may further comprise a heterologous nucleic acid sequence such as a transgene. In some embodiments, the “donor DNA molecule” is an “intervening DNA”.

As used herein, the terms “vicinity”, “vicinity of”, “proximal” or “proximal to” with regard to one or more nucleotide sequences of this invention means immediately next to, or separated by from about 1 base to about 2000 bases (e.g., 1, 2, 3, 4, 5, 6, 7, 8, 9, 10, 15, 20, 30, 40, 50, 100, 200, 250, 300, 350, 400, 450, 500, 750, 1000, 1500 or 2000 bases), including any values included within this range but not explicitly recited herein.

A “microRNA” (abbreviated miRNA) is a small non-coding RNA molecule (containing between about 20 and about 24 nucleotides, generally about 22 nucleotides) found in plants, animals and some viruses, which functions in RNA silencing and post-transcriptional regulation of gene expression. miRNA genes are usually transcribed by RNA polymerase II (Pol II). The polymerase often binds to a promoter found near the DNA sequence, encoding what will become the hairpin loop of the pre-miRNA. The resulting transcript is capped with a specially modified nucleotide at the 5′ end, polyadenylated with multiple adenosines (poly-A tail), and spliced.

“pre-miRNA” is the miRNA precursor with the stem-loop structure with 5′ cap and 3′ ploy-A removed. It's the natural structure helping to produce miRNA. Sometimes this term is used to distinguish it from the mature miRNA (the between about 20 and about 24 nucleotides, generally about 22 nucleotides sequence). In this way is meant the structure, not the final functional short sequence. The terms “miRNA scaffold” or “miRNA backbone” are equally used within the context of the present invention to refer to the pre-miRNA structure.

As used herein, the term “amiRNA” (artificial miRNA) generally refers to a natural miRNA scaffold of which its core sequence (the mature miRNA sequence and corresponding miRNA* sequence) was substituted by an “amiRNA core” sequence to redirect the targeting (silencing) towards a new gene. The term “amiRNA core” refers to the artificial (designed) part of this approach, the about 20 to 24 nucleotide short sequence complementary to the new target gene. In this context, the term complementary refers to the ability of the amiRNA to bind the target RNA molecule. In some embodiments, the amiRNA core is 90% complementary to the new target gene molecule and retains its ability to bind the target RNA molecule.

As used herein, the term “guide RNA” or “gRNA” generally refers to an RNA molecule (or a group of RNA molecules collectively) that can bind to a CRISPR system effector, such as a Cas or a Cpf 1 protein, and aid in targeting the Cas or Cpf 1 protein to a specific location within a target polynucleotide (e.g., a DNA). A guide RNA of the invention can be an engineered, single RNA molecule (sgRNA), where for example the sgRNA comprises a crRNA segment and optionally a tracrRNA segment. A guide RNA of the invention can also be a dual-guide system, where the crRNA and tracrRNA molecules are physically distinct molecules which then interact to form a duplex for recruitment of a CRISPR system effector, such as Cas9, and for targeting of that protein to the target polynucleotide.

As used herein, the term “crRNA” or “crRNA segment” refers to an RNA molecule or to a portion of an RNA molecule that includes a polynucleotide targeting guide sequence, a stem sequence involved in protein-binding, and, optionally, a 3′-overhang sequence. The polynucleotide targeting guide sequence is a nucleic acid sequence that is complementary to a sequence in a target DNA. This polynucleotide targeting guide sequence is also referred to as the “protospacer”. In other words, the polynucleotide targeting guide sequence of a crRNA molecule interacts with a target DNA in a sequence-specific manner via hybridization (i.e., base pairing). As such, the nucleotide sequence of the polynucleotide targeting guide sequence of the crRNA molecule may vary and determines the location within the target DNA that the guide RNA and the target DNA will interact.

The polynucleotide targeting guide sequence of a crRNA molecule can be modified (e.g., by genetic engineering) to hybridize to any desired sequence within a target DNA. The polynucleotide targeting guide sequence of a crRNA molecule of the invention can have a length from about 12 nucleotides to about 100 nucleotides. For example, the polynucleotide targeting guide sequence of a crRNA can have a length of from about 12 nucleotides (nt) to about 80 nt, from about 12 nt to about 50 nt, from about 12 nt to about 40 nt, from about 12 nt to about 30 nt, from about 12 nt to about 25 nt, from about 12 nt to about 20 nt, or from about 12 nt to about 19 nt. For example, the polynucleotide targeting guide sequence of a crRNA can have a length of from about 17 nt to about 27 nts. For example, the polynucleotide targeting guide sequence of a crRNA can have a length of from about 19 nt to about 20 nt, from about 19 nt to about 25 nt, from about 19 nt to about 30 nt, from about 19 nt to about 35 nt, from about 19 nt to about 40 nt, from about 19 nt to about 45 nt, from about 19 nt to about 50 nt, from about 19 nt to about 60 nt, from about 19 nt to about 70 nt, from about 19 nt to about 80 nt, from about 19 nt to about 90 nt, from about 19 nt to about 100 nt, from about 20 nt to about 25 nt, from about 20 nt to about 30 nt, from about 20 nt to about 35 nt, from about 20 nt to about 40 nt, from about 20 nt to about 45 nt, from about 20 nt to about 50 nt, from about 20 nt to about 60 nt, from about 20 nt to about 70 nt, from about 20 nt to about 80 nt, from about 20 nt to about 90 nt, or from about 20 nt to about 100 nt. The nucleotide sequence of the polynucleotide targeting guide sequence of a crRNA can have a length at least about 12 nt. In some embodiments, the polynucleotide targeting guide sequence of a crRNA is 20 nucleotides in length. In some embodiments, the polynucleotide targeting guide sequence of a crRNA is 19 nucleotides in length.

The present invention also provides a guide RNA comprising an engineered crRNA, wherein the crRNA comprises a bait RNA segment capable of hybridizing to a genomic target sequence. This engineered crRNA maybe a physically distinct molecule, as in a dual-guide system.

As used herein, the term “tracrRNA” or “tracrRNA segment” refers to an RNA molecule or portion thereof that includes a protein-binding segment (e.g., the protein-binding segment is capable of interacting with a CRISPR-associated protein, such as a Cas9). The present invention also provides a guide RNA comprising an engineered tracrRNA, wherein the tracrRNA further comprises a bait RNA segment that is capable of binding to a donor DNA molecule. The engineered tracrRNA may be a physically distinct molecule, as in a dual-guide system, or may be a segment of a sgRNA molecule.

In some embodiments, the guide RNA, either as a sgRNA or as two or more RNA molecules, does not contain a tracrRNA, as it is known in the art that some CRISPR-associated nucleases, such as Cpf1 (also known as Cas12a), do not require a tracrRNA for its RNA-mediated endonuclease activity (Qi et al., 2013, Cell, 152: 1173-1183; Zetsche et al., 2015, Cell 163: 759-771). Such a guide RNA of the invention may comprise a crRNA with the bait RNA operably linked at the 5′ or 3′ end of the crRNA. Cpf1 also has RNase activity on its cognate pre-crRNA (Fonfara et al., 2016, Nature, doi.org/10.1038/nature17945). A guide RNA of the invention may comprise multiple crRNAs which the Cpf1 processes to mature crRNAs. In some embodiments, each of these crRNAs is operably linked to a bait RNA. In other embodiments, at least one of these crRNAs is operably linked to a bait RNA. The bait RNA may be specific to a sequence of interest (SOI) or target genomic site, as described in the Examples herein.

The present invention also provides a nucleic acid molecule comprising a nucleic acid sequence encoding a guide RNA of the invention. The nucleic acid molecule may be a DNA or an RNA molecule. In some embodiments, the nucleic acid molecule is circularized. In other embodiments, the nucleic acid molecule is linear. In some embodiments, the nucleic acid molecule is single stranded, partially double-stranded, or double-stranded. In some embodiments, the nucleic acid molecule is complexed with at least one polypeptide. The polypeptide may have a nucleic acid recognition or nucleic acid binding domain. In some embodiments, the polypeptide is a shuttle for mediating delivery of, for example, a chimeric RNA of the invention, a nuclease, and optionally a donor molecule. In some embodiments, the polypeptide is a Feldan Shuttle (U.S. Patent Publication No. 20160298078, herein incorporated by reference). The nucleic acid molecule may comprise an expression cassette capable of driving the expression of the chimeric RNA. The nucleic acid molecule may further comprise additional expression cassettes, capable of expressing, for example, a nuclease such as a CRISPR-associated nuclease. The present invention also provides an expression cassette comprising a nucleic acid sequence encoding a chimeric RNA of the invention.

A “site-directed modifying polypeptide” modifies the target DNA (e.g., cleavage or methylation of target DNA) and/or a polypeptide associated with target DNA (e.g., methylation or acetylation of a histone tail). A site-directed modifying polypeptide is also referred to herein as a “site-directed polypeptide” or an “RNA binding site-directed modifying polypeptide.” The site-directed modifying polypeptide interacts with the guide RNA, which is either a single RNA molecule or a RNA duplex of at least two RNA molecules, and is guided to a DNA sequence (e.g. a chromosomal sequence or an extrachromosomal sequence, e.g. an episomal sequence, a minicircle sequence, a mitochondrial sequence, a chloroplast sequence, etc.) by virtue of its association with the guide RNA.

In some cases, the site-directed modifying polypeptide is a naturally occurring modifying polypeptide. In other cases, the site-directed modifying polypeptide is not a naturally-occurring polypeptide (e.g., a chimeric polypeptide or a naturally-occurring polypeptide that is modified, e.g., mutation, deletion, insertion). Exemplary naturally-occurring site-directed modifying polypeptides are known in the art (see for example, Makarova et al., 2017, Cell 168: 328-328.e1, and Shmakov et al., 2017, Nat Rev Microbiol 15(3): 169-182, both herein incorporated by reference). These naturally occurring polypeptides bind a DNA-targeting RNA, are thereby directed to a specific sequence within a target DNA, and cleave the target DNA to generate a double strand break.

A site-directed modifying polypeptide comprises two portions, an RNA-binding portion and an activity portion. In some embodiments, the site-directed modifying polypeptide comprises: (i) an RNA-binding portion that interacts with a DNA-targeting RNA, wherein the DNA-targeting RNA comprises a nucleotide sequence that is complementary to a sequence in a target DNA; and (ii) an activity portion that exhibits site-directed enzymatic activity (e.g., activity for DNA methylation, activity for DNA cleavage, activity for histone acetylation, activity for histone methylation, etc.), wherein the site of enzymatic activity is determined by the DNA-targeting RNA. In other embodiments, a site-directed modifying polypeptide comprises: (i) an RNA-binding portion that interacts with a DNA-targeting RNA, wherein the DNA-targeting RNA comprises a nucleotide sequence that is complementary to a sequence in a target DNA; and (ii) an activity portion that modulates transcription within the target DNA (e.g., to increase or decrease transcription), wherein the site of modulated transcription within the target DNA is determined by the DNA-targeting RNA.

In some cases, the site-directed modifying polypeptide has enzymatic activity that modifies target DNA (e.g., nuclease activity, methyltransferase activity, demethylase activity, DNA repair activity, DNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity or glycosylase activity). In other cases, the site-directed modifying polypeptide has enzymatic activity that modifies a polypeptide (e.g., a histone) associated with target DNA (e.g., methyltransferase activity, demethylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity or demyristoylation activity).

In some cases, different site-directed modifying polypeptides, for example different Cas9 proteins (i.e., Cas9 proteins from various species) may be advantageous to use in the various provided methods of the invention to capitalize on various enzymatic characteristics of the different Cas9 proteins (e.g., for different Protospacer Adjacent Motif (PAM) sequence preferences; for increased or decreased enzymatic activity; for an increased or decreased level of cellular toxicity; to change the balance between NHEJ, homology-directed repair, single strand breaks, double strand breaks, etc.). Cas9 proteins from various species (for example, those disclosed in Shmakov et al., 2017, or polypeptides derived therefrom) may require different PAM sequences in the target DNA. Thus, for a particular Cas9 enzyme of choice, the PAM sequence requirement may be different than the 5′-N GG-3′ sequence (where N is either a A, T, C, or G) known to be required for Cas9 activity. Many Cas9 orthologues from a wide variety of species have been identified herein and the proteins share only a few identical amino acids. All identified Cas9 orthologs have the same domain architecture with a central HNH endonuclease domain and a split RuvC/RNaseH domain. Cas9 proteins share 4 key motifs with a conserved architecture; Motifs 1, 2, and 4 are RuvC like motifs, while motif 3 is an HNH-motif.

The site-directed modifying polypeptide may also be a chimeric and modified Cas9 nuclease. For example, it may be a modified Cas9 “base editor”. Base editing enables direct, irreversible conversion of one target DNA base into another in a programmable manner, without requiring DNA cleavage or a donor DNA molecule. For example, Komor et al (2016, Nature, 533: 420-424), teach a Cas9-cytidine deaminase fusion, where the Cas9 has also been engineered to be inactivated and not induce double-stranded DNA breaks. Additionally, Gaudelli et al (2017, Nature, doi:10.1038/nature24644) teach a catalytically impaired Cas9 fused to a tRNA adenosine deaminase, which can mediate conversion of an A/T to G/C in a target DNA sequence. Another class of engineered Cas9 nucleases which may act as a site-directed modifying polypeptide in the methods and compositions of the invention are variants which can recognize a broad range of PAM sequences, including NG, GAA, and GAT (Hu et al., 2018, Nature, doi:10.1038/nature26155).

Any Cas9 protein, including those naturally occurring and/or those mutated or modified from naturally occurring Cas9 proteins, can be used as a site-directed modifying polypeptide in the methods and compositions of the present invention. Catalytically active Cas9 nucleases cleave target DNA to produce double strand breaks. These breaks are then repaired by the cell in one of two ways: non-homologous end joining, and homology-directed repair.

In non-homologous end joining (NHEJ), the double-strand breaks are repaired by direct ligation of the break ends to one another. As such, no new nucleic acid material is inserted into the site, although some nucleic acid material may be lost, resulting in a deletion. In homology-directed repair, a donor DNA molecule or an intervening DNA with homology to the cleaved target DNA sequence is used as a template for repair of the cleaved target DNA sequence, resulting in the transfer of genetic information from the donor polynucleotide to the target DNA. As such, new nucleic acid material may be inserted/copied into the site. In some cases, a target DNA is contacted with a donor molecule, for example a donor DNA molecule or an intervening DNA molecule. In some cases, a donor DNA molecule or an intervening DNA molecule is introduced into a cell. In some cases, at least a segment of a donor DNA molecule or an intervening DNA molecule integrates into the genome of the cell.

The modifications of the target DNA due to NHEJ and/or homology-directed repair lead to, for example, gene correction, gene replacement, gene tagging, transgene insertion, nucleotide deletion, gene disruption, gene mutation, etc. Accordingly, cleavage of DNA by a site-directed modifying polypeptide may be used to delete nucleic acid material from a target DNA sequence (e.g., to disrupt a gene that makes cells susceptible to infection (e.g. the CCRS or CXCR4 gene, which makes T cells susceptible to HIV infection), to remove disease-causing trinucleotide repeat sequences in neurons, to create gene knockouts and mutations as disease models in research, etc.) by cleaving the target DNA sequence and allowing the cell to repair the sequence in the absence of an exogenously provided donor polynucleotide. Thus, the subject methods can be used to knock out a gene (resulting in complete lack of transcription or altered transcription) or to knock in genetic material into a locus of choice in the target DNA. Alternatively, if a DNA-targeting RNA duplex and a site-directed modifying polypeptide are co-administered to cells with a donor molecule that includes at least a segment with homology to the target DNA sequence, the subject methods may be used to add, i.e. insert or replace, nucleic acid material to a target DNA sequence (e.g. to “knock in” a nucleic acid that encodes for a protein, an siRNA, an miRNA, etc.), to add a tag (e.g., 6× His, a fluorescent protein (e.g., a green fluorescent protein; a yellow fluorescent protein, etc.), hemagglutinin (HA), FLAG, etc.), to add a regulatory sequence to a gene (e.g. promoter, polyadenylation signal, internal ribosome entry sequence (IRES), 2A peptide, start codon, stop codon, splice signal, localization signal, etc.), to modify a nucleic acid sequence (e.g., introduce a mutation), and the like. As such, a complex comprising a DNA-targeting RNA duplex and a site-directed modifying polypeptide is useful in any in vitro or in vivo application in which it is desirable to modify DNA in a site-specific, i.e. “targeted”, way, for example gene knock-out, gene knock-in, gene editing, gene tagging, etc., as used in, for example, gene therapy, e.g. to treat a disease or as an antiviral, antipathogenic, or anticancer therapeutic, the production of genetically modified organisms in agriculture, the large scale production of proteins by cells for therapeutic, diagnostic, or research purposes, the induction of iPS cells, biological research, the targeting of genes of pathogens for deletion or replacement, etc.

The term “CRISPR-associated protein”, “Cas protein”, “CRISPR-associated nuclease” or “Cas nuclease” refers to a wild type Cas protein, a fragment thereof, or a mutant or variant thereof. The term “Cas mutant” or “Cas variant” refers to a protein or polypeptide derivative of a wild type Cas protein, e.g., a protein having one or more point mutations, insertions, deletions, truncations, a fusion protein, or a combination thereof. In certain embodiments, the Cas mutant or Cas variant substantially retains the nuclease activity of the Cas protein, such as a Cas9 variant described herein which is operably linked to a nuclear localization signal (NLS) derived from a plant. In certain embodiments, the Cas nuclease is mutated such that one or both nuclease domains are inactive, such as, for example, a catalytically dead Cas9 referred to as dCas9, which is still able to target to a specific genomic location but has no endonuclease activity (Qi et al., 2013, Cell, 152: 1173-1183, hereby incorporated within). In some embodiments, the Cas nuclease is mutated so that it lacks some or all of the nuclease activity of its wild-type counterpart. The Cas protein may be Cas9, Cpf 1 (Zetsche et al., 2015, Ce11,163: 759-771, hereby incorporated within) or any another CRISPR-associated nuclease.

Argonaute proteins from bacteria such as Thermus thermophilus can also be used as genome editing in a similar manner to CRISPR/Cas9. Similar to Cas9, Argonaute proteins are believed to use oligonucleotides as guides to degrade invasive genomes. The complex of these guides and the Thermus thermophilus Argonaut protein cleave complementary DNA strands at high temperature (75 degrees C.). WO 2014/189628 describes one way in which this system could be used for genome editing. Additional examples include WO2014/189628, WO2016/161375, and WO2016/166268.

The present invention provides a method of reducing expression of a target gene comprised of, introducing into a plant cell a nuclease capable of site-directed DNA cleavage at a genomic site encoding a native pre-miRNA of said plant cell, making at least one double strand break at said genomic site or in the vicinity of said genomic site, selecting for a cell where said at least one double strand break has been repaired with an intervening DNA replacing said genomic site, and reducing expression of the target gene, wherein said intervening DNA encodes a modified pre-miRNA comprising an amiRNA core sequence complementary to said target gene.

The genomic site encodes a native pre-miRNA of the plant cell which is being modified by the methods of the present invention. The intervening DNA is a piece of DNA identical to the genomic site encoding a native pre-miRNA of the plant cell but with the replacement of the native miRNA core sequence with the amiRNA core sequence complementary to the new target gene. The intervening DNA is introduced into the plant cell together with the nuclease.

In a further embodiment, the invention relates to the method of any of the preceding embodiments wherein the nuclease capable of site-directed DNA cleavage at a genomic site encoding a native pre-miRNA makes one double strand break at said genomic site sequence.

In a further embodiment, the invention relates to the method of the preceding embodiment wherein the nuclease capable of site-directed DNA cleavage at a genomic site encoding a native pre-miRNA makes one double strand break in the vicinity of said genomic site, preferably within 2 kb upstream or downstream of said genomic site.

In a further embodiment, the invention relates to the method of the preceding embodiment wherein the nuclease capable of site-directed DNA cleavage at a genomic site encoding a native pre-miRNA makes one double strand break in the vicinity of said genomic site, preferably within 500 nucleotides upstream or downstream of said genomic site.

In a further embodiment, the invention relates to the method of the preceding embodiment wherein the nuclease capable of site-directed DNA cleavage at a genomic site encoding a native pre-miRNA makes one double strand break within 100 nucleotides upstream or downstream of said genomic site.

In a further embodiment, the invention relates to the method of the preceding embodiment wherein the nuclease capable of site-directed DNA cleavage at a genomic site encoding a native pre-miRNA of said plant cell makes at least two double strand breaks at said genomic site or in the vicinity of said genomic site.

In a further embodiment, the invention relates to the method of the preceding embodiment wherein the target gene is an exogenous target gene, more preferably a pest gene, more preferably a viral, fungal or microbial gene.

In a further embodiment, the invention relates to the method of any of the preceding embodiments wherein the target gene is an insect pest gene or a nematode pest gene.

In a further embodiment, the invention relates to the method of any one of the preceding embodiments, wherein the target gene is a Bunyavirales gene, preferably a tospovirus gene, more preferably a tomato spotted wilt virus (TSWV) gene.

In a further embodiment, the invention relates to the method of any one of the preceding embodiments, wherein the target gene is an endogenous plant gene.

In a further embodiment, the invention relates to the method of any one of the preceding embodiments, wherein the target endogenous plant gene is a gene involved in plant development, biotic or abiotic stress.

In a further embodiment, the invention relates to the method of any one of the preceding embodiments, wherein said plant cell is a Solanaceae, corn, rice, canola, soybean or sunflower cell. In a further embodiment, the invention relates to the method of any one of the preceding embodiments, wherein said plant cell is a tomato cell.

In a further embodiment, the invention relates to the method of any one of the preceding embodiments, wherein said genomic site encoding a native pre-miRNA encodes a native tomato pre-miRNA.

In a further embodiment, the invention relates to the method of any one of the preceding embodiments, wherein said genomic site comprises SEQ ID NO:6 or SEQ ID

NO:7.

In a further embodiment, the invention relates to the method of any one of the preceding embodiments, wherein said genomic site consists of SEQ ID NO:6 or SEQ ID NO:7.

In a further embodiment, the invention relates to the method of any one of the preceding embodiments, wherein said genomic site encodes the SlmiR156b or SlmiR1919b gene.

In a further embodiment, the invention relates to the method of any one of the preceding embodiments, wherein the intervening DNA comprises any one of SEQ ID NOs: 1 to 5.

In a further embodiment, the invention relates to the method of any one of the preceding embodiments, wherein the intervening DNA comprises any one of SEQ ID NOs: 22 to 24.

In a further embodiment, the invention relates to the method of any one of the preceding embodiments, wherein the intervening DNA comprises any one of SEQ ID NOs:

8 to 17.

In a further embodiment, the invention relates to the method of any one of the preceding embodiments, wherein said nuclease is selected from the group consisting of meganucleases (MNs), zinc-finger nucleases (ZFNs), transcription-activator like effector nucleases (TALENs), Cas9 nuclease, Cfp1 nuclease, dCas9-Fokl, dCpf1-Fokl, chimeric Cas9/Cpf1-cytidine deaminase, chimeric Cas9/Cpf1-adenine deaminase, chimeric FEN1-Fokl, and Mega-TALs, a nickase Cas9 (nCas9), chimeric dCas9 non-Fokl nuclease and dCpf 1 non-Fokl nuclease.

In a further embodiment, the invention relates to the method of any one of the preceding embodiments, wherein said cell has a haploid, diploid, polyploid, or hexiploid genome.

In a further embodiment, the invention relates to the method of any one of the preceding embodiments, wherein said cell is heterozygous for the modified pre-miRNA.

In a further embodiment, the invention relates to the method of any one of the preceding embodiments, wherein said cell has one copy of the modified pre-miRNA and one copy of the native pre-miRNA.

In the context of the present invention, a haploid plant cell comprising one copy of the modified pre-miRNA has utility in e.g. breeding processes and methods for seed production.

In a further embodiment, the invention relates to a method of producing plant seeds, preferably Solanaceae, corn, rice, canola, soybean or sunflower seeds, more preferably tomato seeds, comprising crossing a plant comprising a plant cell obtained by the method of any one of the preceding embodiments with itself or with another plant of the same crop.

In a further embodiment, the invention relates to the method of any one of the preceding embodiments, wherein said method additionally comprises the use of one or more guide sequences. In a further embodiment, the invention relates to the method of any one of the preceding embodiments, wherein one or more guide sequences are introduced into the cell together with said nuclease. In a further embodiment, the invention relates to the method of any one of the preceding embodiments, wherein one or more guide sequences are derived from the target genomic site.

In a further embodiment, the method of any of the preceding embodiments confers resistance to a plant pest.

In a further embodiment, the invention relates to a plant cell, preferably a

Solanaceae, corn, rice, canola, soybean or sunflower cell, more preferably a tomato plant cell obtained by the method of any one of the preceding embodiments.

In a further embodiment, the invention relates to the plant cell of the preceding embodiment, wherein said cell comprises any one of SEQ ID NOs: 1-5.

In a further embodiment, the invention relates to the plant cell of the preceding embodiment, wherein said cell comprises any one of SEQ ID NOs: 22-24.

In a further embodiment, the invention relates to the plant cell of the preceding embodiment, wherein said cell comprises any one of SEQ ID NOs: 8-17.

In a further embodiment, the invention relates to a plant cell comprising any one of SEQ ID NOs: 1-5.

In a further embodiment, the invention relates to a plant cell comprising any one of SEQ ID NOs: 22-24.

In a further embodiment, the invention relates to a plant cell comprising any one of SEQ ID NOs: 8-17.

In a further embodiment, the invention relates to a diploid plant cell comprising one copy of SEQ ID NO:6 and one copy of any one of SEQ ID NOs: 8-12.

In a further embodiment, the invention relates to a diploid plant cell comprising one copy of SEQ ID NO:7 and one copy of any one of SEQ ID NOs: 13-17.

In a further embodiment, the invention relates to a method of producing plant seeds, preferably Solanaceae, corn, rice, canola, soybean or sunflower seeds, more preferably tomato seeds, comprising crossing a plant comprising a plant cell according to any one of the preceding embodiments with itself or with another plant of the same crop.

In a further embodiment, the invention relates to a plant comprising a plant cell according to any of the preceding embodiments. In a further embodiment, the invention relates to a tomato plant comprising a plant cell according to any of the preceding embodiments.

In a further embodiment, the invention relates to a plant part comprising a plant cell according to any of the preceding embodiments. In a further embodiment, the invention relates to a tomato plant part comprising a plant cell according to any of the preceding embodiments. In a further embodiment, the plant part is a plant seed, preferably a tomato plant seed.

In a further embodiment, the plant or plant part according to any of the preceding embodiments provides pest resistance. In a further embodiment, the plant or plant part according to any of the preceding embodiments provides pest resistance towards tospoviruses. In a further embodiment, the plant or plant part according to any of the preceding embodiments provides resistance towards TSWV.

In a further embodiment, the invention relates to a method of producing plant seeds, preferably Solanaceae, corn, rice, canola, soybean or sunflower seeds, more preferably tomato seeds, comprising crossing a plant according to any one of the preceding embodiments with itself or with another plant of the same crop.

In a further embodiment, the invention relates to a method of producing a plant, preferably a Solanaceae, corn, rice, canola, soybean or sunflower plant, more preferably a tomato plant, comprising crossing a plant according to any one of the preceding embodiments with itself or with another plant of the same crop to produce a progeny plant comprising the amiRNA of the present invention and exhibiting a novel phenotype.

The methods of the present invention have been practiced and exemplified with the model crop tomato and the model virus tomato spotted wilt virus (TSWV). The skilled person with the information disclosed herein can easily transfer the knowledge and carry out the methods of the present invention in different plants and with different target types.

### EXAMPLES

### Example 1: Identification of TSWV Sequences Suitable to be Used as amiRNA Core

Published TSWV genomes were collected (Table 1) and aligned.

Table 1 lists the TSWV genomes collected from NCBI (found on the World Wide Web at www.ncbi.nlm.nih.gov/nuccore/).

| length | db | acc. | species | seq. type | isolate |
|---|---|---|---|---|---|
| 2899 | gb | KM365066.1 | TSWV | complete sequence | WA7 |
| 2920 | gb | JN664252.1 | TSWV | complete sequence | CG-1 |
| 2920 | gb | AY744473.1 | TSWV | complete sequence | CA-6 |
| 2921 | gb | AY744472.1 | TSWV | complete sequence | CA-5 |
| 2921 | gb | AY744471.1 | TSWV | complete sequence | CA-4 |
| 2921 | gb | AY744470.1 | TSWV | complete sequence | CA-3 |
| 2922 | gb | AY744479.1 | TSWV | complete sequence | SPAIN-1 |
| 2923 | gb | AY744480.1 | TSWV | complete sequence | SPAIN-2 |
| 2923 | gb | AY744475.1 | TSWV | complete sequence | CO |
| 2924 | gb | KP008129.1 | TSWV | complete sequence | LL-N.05 |
| 2926 | gb | HQ839729.1 | TSWV | complete sequence | p105-RB-Mar |
| 2926 | gb | AY744469.1 | TSWV | complete sequence | CA-2 |
| 2927 | gb | HQ839731.1 | TSWV | complete sequence | p105-RB-MaxII |
| 2927 | gb | HQ839730.1 | TSWV | complete sequence | p105-RB-MaxI |
| 2927 | gb | AY744474.1 | TSWV | complete sequence | CA-7 |
| 2927 | gb | AY744468.1 | TSWV | complete sequence | CA-1 |
| 2951 | gb | KP008134.1 | TSWV | complete sequence | PVR |
| 2952 | gb | KU179515.1 | TSWV | complete sequence | BasC |
| 2954 | gb | AY744478.1 | TSWV | complete sequence | NC-3 |
| 2959 | gb | AY744476.1 | TSWV | complete sequence | NC-1 |
| 2961 | gb | KP008131.1 | TSWV | complete sequence | Pujol1TL3 |
| 2961 | gb | KC261967.1 | TSWV | complete sequence | TSWV-12 |
| 2961 | gb | KC261973.1 | TSWV | complete sequence | TSWV-17 |
| 2962 | gb | HQ830186.1 | TSWV | complete sequence | p202/3RB |
| 2963 | gb | HQ830187.1 | TSWV | complete sequence | p202/3WT |
| 2967 | gb | KC261958.1 | TSWV | complete sequence | TSWV-7 |
| 2968 | gb | KM657114.1 | TSWV | complete sequence | YNrp |
| 2968 | gb | HM581936.1 | TSWV | complete sequence | Tomato |
| 2969 | gb | KC261955.1 | TSWV | complete sequence | TSWV-6 |
| 2970 | gb | KU976396.1 | TSWV | complete sequence | TSWV-LE |
| 2970 | gb | KU356854.1 | TSWV | complete sequence | TSWV-Celery |
| 2970 | gb | KM657115.1 | TSWV | complete sequence | YNta |
| 2970 | gb | JF960235.1 | TSWV | complete sequence | TSWV-YN |
| 2971 | dbj | LC273307.1 | TSWV | complete sequence | TSWV-HJ |
| 2971 | gb | KM657116.1 | TSWV | complete sequence | YNgp |
| 2971 | gb | KC261949.1 | TSWV | complete sequence | TSWV-4 |
| 2971 | gb | HQ402595.1 | TSWV | complete sequence | KM-T |
| 2973 | gb | KM076653.1 | TSWV | complete sequence | LS3 |
| 2973 | gb | KC261970.1 | TSWV | complete sequence | TSWV-16 |
| 2975 | gb | KC261952.1 | TSWV | complete sequence | TSWV-5 |
| 2975 | gb | KC261964.1 | TSWV | complete sequence | TSWV-10 |
| 2977 | gb | KC261961.1 | TSWV | complete sequence | TSWV-8 |
| 2984 | gb | KT160282.1 | TSWV | complete sequence | — |
| 2991 | dbj | AB190819.1 | TSWV | complete sequence | — |
| 3003 | gb | KT717693.1 | TSWV | complete sequence | TSWV-QLD1 |
| 3013 | gb | HM581942.1 | TSWV | complete sequence | Pepper2 |
| 3013 | gb | HM581939.1 | TSWV | complete sequence | Pepper1 |
| 3014 | gb | KT452079.1 | TSWV | genomic sequence | TSWV-Pt-VA |
| 3016 | gb | AY870392.1 | TSWV | complete sequence | T |
| 3020 | gb | KC261976.1 | TSWV | complete sequence | TSWV-18 |
| 3021 | gb | AY744477.1 | TSWV | complete sequence | NC-2 |
| 3047 | gb | AY870391.1 | TSWV | complete sequence | M |

The conserved TSWV regions with high similarity were selected. 21-nt sequences were analysed for GC contents, secondary structure, specific position and off-target within tomato plant genome (TSWV 21-nt sequences versus tomato genome). TSWV sequences with 30˜60% GC contents and no hits of less than 3 mismatches on the tomato genome were preferred.

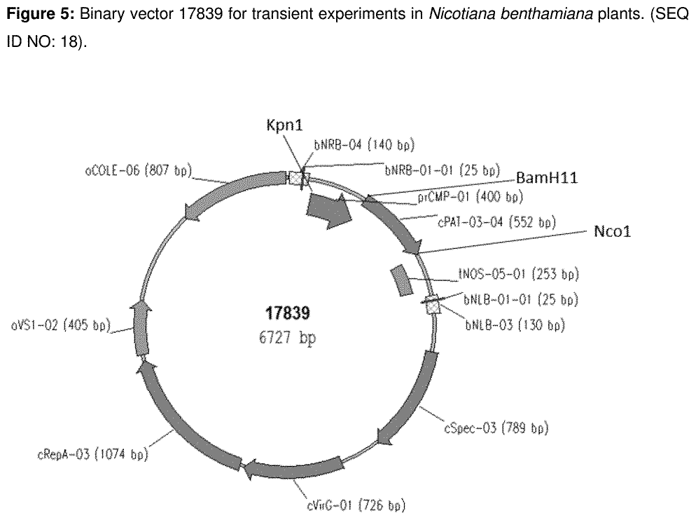

To test whether a given amiRNA core viral sequence can be effective in controlling the virus, potential targets were identified in the TSWV viral genome as described above and tested in transient experiments. Arabidopsis native pre-miRNA AtmiR159a was used as a scaffold. Modified miRNAs were directly synthesized by replacing the native AtmiR159a core sequence with the designed 21-nt sequences complementary to the TSWV target genes. The modified miRNAs were compared with the native miRNA on structure and stability (MFE) and those miRNAs with minimal changes were selected for experimental evaluation and validation in transient virus assays. For these transient assays, binary vector 17839 (FIG. 5) was used to express the designed amiRNA. Both binary vector 17839 and synthetic AtmiR159a-amiRNA fragment was cut out by BamHI/NcoI and gel purified. The two fragments were ligated together and transformed into DH5alpha cell. Positive clones were verified by BamHI/NcoI digestion and all junctions were sequenced.

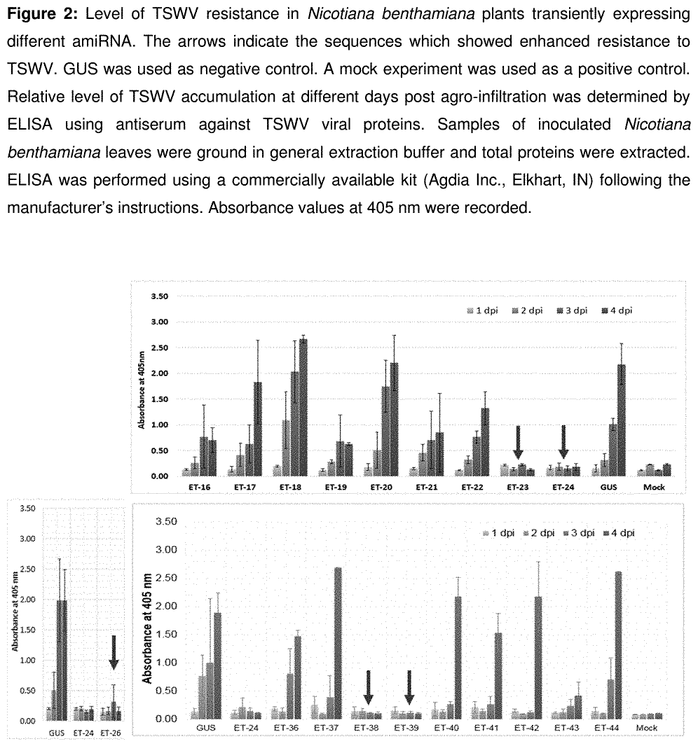

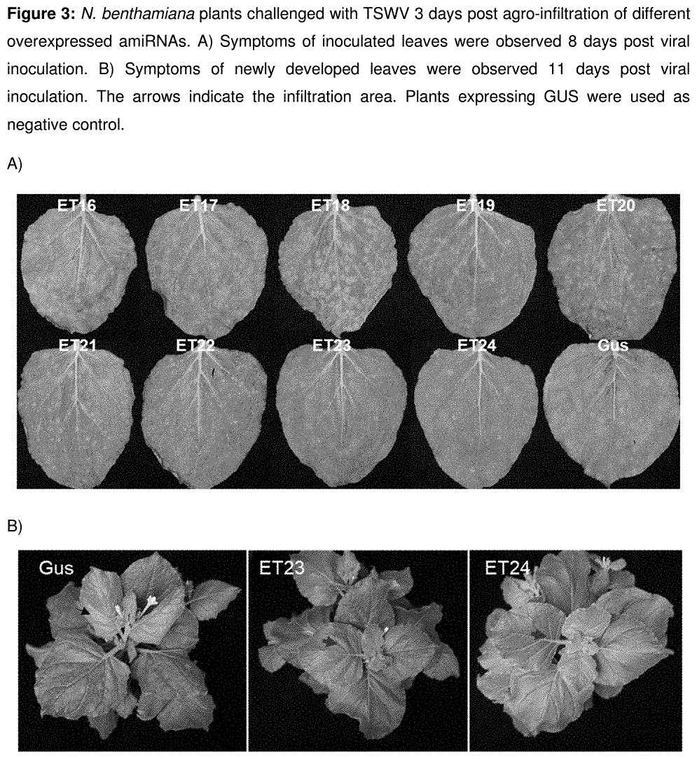

Table 2 lists all TSWV sequences tested as amiRNA core within the AtmiR159a scaffold. Five of them (SEQ ID NOs: 1-5) have been identified as suitable to provide high resistance to TSWV in transient assays (FIGS. 2 and 3).

| | | | SEQ |
|---|---|---|---|
| Lead | amiRNA core | Resistance Efficacy | ID NO: |
| ET-16 | amiRNA_RdRp_GC52 | Intermediate Resistance | |
| ET-17 | amiRNA_RdRp_GC42 | Susceptibility | |
| ET-18 | amiRNA_NSs_GC52 | Susceptibility | |
| ET-19 | amiRNA_N_GC42 | Intermediate Resistance | |
| ET-20 | amiRNA_GnGc_GC52 | Susceptibility | |
| ET-21 | amiRNA_GnGc_GC40 | Intermediate Resistance | |
| ET-22 | amiRNA_NSm_GC30 | Intermediate Resistance | |
| ET-23 | amiTSWV_N1w_PC | High Resistance | 1 |
| ET-24 | amiTSWV_N2_PC | High Resistance | 2 |
| ET-26 | amiTSWV_N2_PC_rev | High Resistance | 3 |
| ET-27 | amiRNA_NSs_GC52_rev | Susceptibility | |
| ET-36 | amiR159a_3p_N_GC42 | Susceptibility | |
| ET-37 | amiR159a_3p_N_GC25 | Susceptibility | |
| ET-38 | amiR159a_3p_N_GC35 | High Resistance | 4 |
| ET-39 | amiR159a_3p_N_GC50 | High Resistance | 5 |
| ET-40 | amiR159a_3p_N_GC43 | Susceptibility | |
| ET-41 | amiR159a_3p_NSs_GC35 | Susceptibility | |
| ET-42 | amiR159a_3p_RdRP_GC25 | Susceptibility | |
| ET-43 | amiR159a_3p_GnGc_GC30 | Intermediate Resistance | |
| ET-44 | amiR159a_3p_NSm_GC40 | Susceptibility | |

Leads ET-23, ET-24, ET-26, ET-38 and ET-39 provide a high level of resistance against TSWV. This approach described in Example 1 therefore allows to identify suitable amiRNA core sequences which are homologous to new target genes and can effectively be used to obtain novel phenotypes. It is noteworthy that ET-26, the reverse-complement sequence of ET-24, also provides a high level of resistance, indicating that once an effective amiRNA core sequence is identified, its reverse-complement sequence can also be used successfully using the methods of the invention.

### Example 2: Identification of Suitable Native Tomato Pre-miRNA Sequences

To test whether a given native tomato pre-miRNA sequence can be effectively used as a receptacle of TSWV amiRNA core sequences for controlling viruses, potential pre-miRNA scaffolds were identified in the tomato genome and tested using ET-24 (SEQ ID NO: 2) as the TSWV amiRNA core sequence (see Example 1).

Published tomato sRNA-seq data were collected (Table 3) to check native miRNA expression.

Table 3 lists the tomato sRNA-seq datasets collected from NCBI SRA database (found on World Wide Web at www.ncbi.nlm.nih.gov/sra/).

| Run | Exp. | Length | Total Spots |
|---|---|---|---|
| SRR039920 | SRX019222 | 36 | 5299195 |
| SRR039921 | SRX019223 | 36 | 4574008 |
| SRR2039800 | SRX1038192 | 37 | 6202076 |
| SRR2989577 | SRX1478064 | 36 | 11026240 |
| SRR2989578 | SRX1478065 | 36 | 18528550 |
| SRR4013313 | SRX2008739 | 50 | 23760631 |
| SRR4346447 | SRX2213272 | 51 | 46872476 |
| SRR5031857 | SRX2356906 | 51 | 2655264 |
| SRR5031858 | SRX2356907 | 51 | 4954975 |
| SRR5031859 | SRX2356908 | 51 | 4375546 |
| SRR786979 | SRX252396 | 36 | 15573561 |
| SRR786980 | SRX252397 | 36 | 13077046 |
| SRR1463412 | SRX627473 | 49 | 18158256 |
| SRR1777738 | SRX833690 | 50 | 10309183 |
| SRR1795959 | SRX871216 | 51 | 73080323 |

The mature miRNAs abundance were analysed throughout these datasets and compared with published data on miRBase (found on the World Wide Web at www.mirbase.org/). The following criteria were used to select tomato native miRNA for modification, including miRNA with multiple family members, producing identical mature miRNA and high expression level especially in green tissues.

Some good candidates listed in Table 3 were selected for further experiments. The amiRNA core sequence ET24 (SEQ ID NO: 2) was first used to validate these candidates and later on new 21-nt sequences were used too. Binary Vector 17839 was firstly digested by Kpn1/Nco1 and a 5762bp fragment was gel purified. The 1 kb promoter region together with the modified pre-miRNA (the miRNA core sequences was replaced by the identified amiRNA core sequence ET-24) were directly synthesized and cut by Kpn1/Nco1. The two fragments were ligated together and transformed into DH5alpha cell. Positive clones were verified by Kpn1/Nco1digestion and all junctions were sequenced.

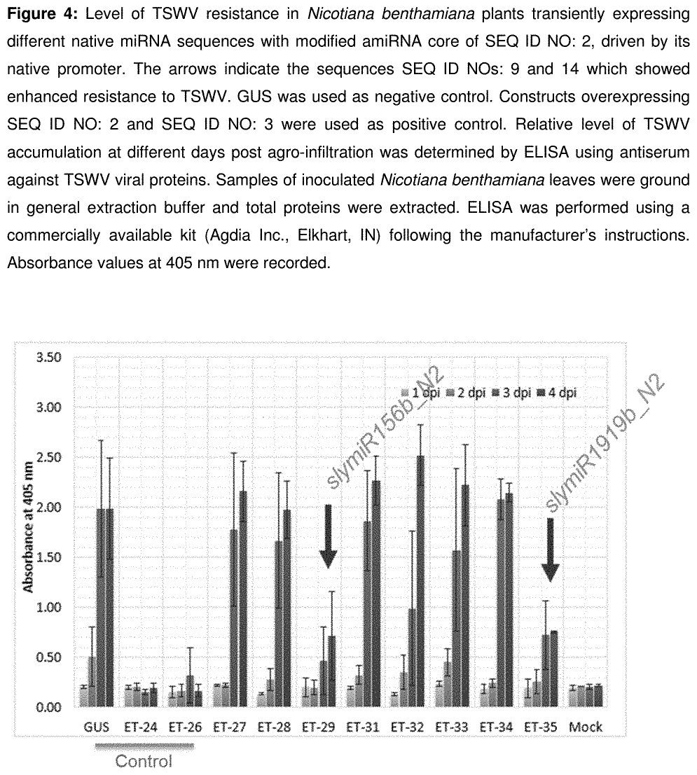

Table 4 lists all sequences tested as pre-miRNA scaffolds. Two of them (SEQ ID NOs: 9 and 14) have been identified as suitable to provide high resistance to TSWV in transient assays (FIG. 4).

| Lead | pre-miRNA | Resistance Efficacy | SEQ ID NO: |
|---|---|---|---|
| ET-28 | miR156a_N2_PC | Susceptibility | |
| ET-29 | miR156b_N2_PC | Resistance | 9 |
| ET-30 | miR168a_N2_PC | NA | |
| ET-31 | miR168b_N2_PC | Susceptibility | |
| ET-32 | miR172a_N2_PC | Susceptibility | |
| ET-33 | miR395b1_N2_PC | Susceptibility | |
| ET-34 | miR395b2_N2_PC | Susceptibility | |
| ET-35 | miR1919b_N2_PC | Resistance | 14 |

The tomato pre-miRNA scaffolds ET-29 and ET-35 holding the amiRNA core TSWV sequence ET-24 (SEQ ID Nos 9 and 14 respectively) show good level of resistance against TSWV, indicating their suitability for being used in the methods of the invention.

### Example 3: Designing Genome Editing Constructs to Modify a Native Tomato Pre-miRNA to Target Tomato Viral Pathogen Gene Targets by Replacing the amiRNA Core Sequence

To test whether editing tomato native miRNA to target viral genes could give tomato resistance to that virus, the following construct was designed to edit the native tomato miRNA SlmiR156b. The target viral genes that were tested are RNA dependent RNA polymerase (RdRp), glycoprotein precursor (Gn/Gc), non-structural movement protein (NSm), non-structural silencing suppressor protein (NSs) and nucleocapsid protein (N) from TSWV. Cas9 was used with two gRNAs to generate double-strand breaks around the tomato native SlmiR156b locus and provide for a modified amiRNA donor for replacement.

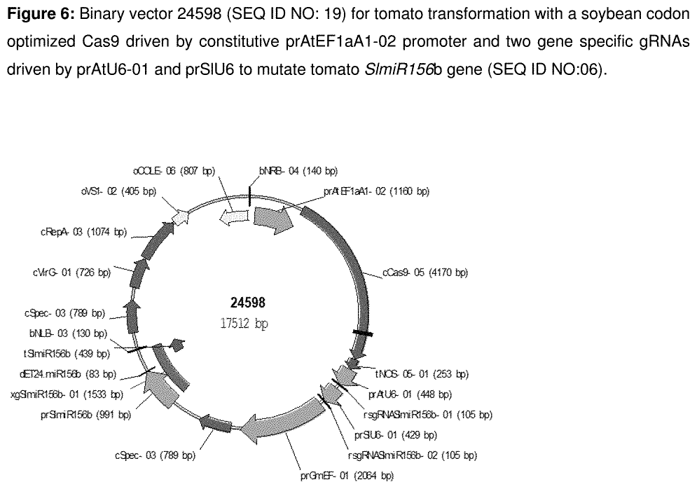

Binary vector 24598 (FIG. 6) for tomato transformation contains soybean codon optimized Cas9 driven by constitutive prAtEF1aA1-02 promoter and two gene specific gRNAs driven by prAtU6-01 and prSIU6 to edit tomato SlmiR156b gene. This construct is to replace native SlmiR156b core sequence by artificial core sequence which targets the TSWV viral genome. A 1.5 kb donor sequence containing 1 kb promoter, pre-SlmiR156b with artificial core and 0.5 kb terminator was also included. cSpec-03 driven by prGmEF-01 is used as selectable marker. The donor DNA fragment and the two gRNAs cassettes of prAtU6-01-rsgRNASImiR156b-A (SEQ ID NO: 20) and prSIU6-rsgRNASImiR156b-B (SEQ ID NO: 21) were synthesized by Generalbiol. All the four cassettes in this binary vector were part of a single transgene.

1) A method of reducing expression of a target gene comprised of:
a) introducing into a plant cell a nuclease capable of site-directed DNA cleavage at a genomic site encoding a native pre-miRNA of said plant cell;
b) making at least one double strand DNA break at said genomic site or in the vicinity of said genomic site;
c) selecting for a cell where said at least one double strand break has been repaired with an intervening DNA replacing said genomic site;
d) reducing expression of the target gene;
wherein said intervening DNA encodes a modified pre-miRNA comprising an amiRNA core sequence complementary to said target gene.

2) The method of claim 1 wherein the target gene is an exogenous target gene, more preferably a pest gene, more preferably a viral, fungal or microbial gene.

3) The method of claim 1 wherein the target gene is a Bunyavirales gene, preferably a tospovirus gene, more preferably a tomato spotted wilt virus gene.

4) The method of claim 1, wherein the target gene is an endogenous plant gene.

5) The method of claim 4, wherein the target endogenous plant gene is a gene involved in plant development, biotic or abiotic stress.

6) The method of claim 1 wherein said plant cell is a Solanaceae, corn, rice, canola, soybean or sunflower cell.

7) The method of claim 1 wherein said cell is a tomato cell.

8) The method of claim 1 wherein said genomic site encoding a native pre-miRNA encodes a native tomato pre-miRNA.

9) The method of claim 1 wherein said genomic site comprises SEQ ID NO: 6 or SEQ ID NO: 7.

10) The method of claim 1 wherein said intervening DNA comprises any one of SEQ ID NOs: 1 to 5.

11) The method of any one claim 1, wherein said nuclease is selected from the group consisting of meganucleases (MNs), zinc-finger nucleases (ZFNs), transcription-activator like effector nucleases (TALENs), Cas9 nuclease, Cfp1 nuclease, dCas9-Fokl, dCpf1-Fokl, chimeric Cas9/Cpf1-cytidine deaminase, chimeric Cas9/Cpf1-adenine deaminase, chimeric FEN1-Fokl, and Mega-TALs, a nickase Cas9 (nCas9), chimeric dCas9 non-Fokl nuclease and dCpf1 non-Fokl nuclease.

12) The method of claim 1 wherein said cell has a haploid, diploid, polyploid, or hexiploid genome.

13) The method of claim 1 wherein said cell is heterozygous for the modified pre-miRNA.

14) The method of claim 1, wherein one or more guide sequences are introduced together with said nuclease.

15) A plant cell, preferably a Solanaceae, corn, rice, canola, soybean or sunflower cell, more preferably a tomato plant cell obtained by the method of claim 1.

16) The cell of claim 15 comprising any one of SEQ ID NOs: 1-5.

17) The cell of claim 16 comprising any one of SEQ ID NOs: 8-17.

18) A method of producing plant seeds, preferably Solanaceae, corn, rice, canola, soybean or sunflower seeds, more preferably tomato seeds, comprising crossing a plant comprising the plant cell obtained by the method of claim 1 with itself or with another plant of the same crop.


SEQUENCE LISTING (24 sequences)

SEQ ID NO: 1 - DNA, 21 bp, Tomato spotted wilt tospovirus
cagtgttgtctgtgctatata

SEQ ID NO: 2 - DNA, 21 bp, Tomato spotted wilt tospovirus
atgaaatgttcggggttaaaa

SEQ ID NO: 3 - DNA, 21 bp, Tomato spotted wilt tospovirus
ttttaaccccgaacatttcat

SEQ ID NO: 4 - DNA, 21 bp, Tomato spotted wilt tospovirus
ttcaaatgctttgcttttcag

SEQ ID NO: 5 - DNA, 21 bp, Tomato spotted wilt tospovirus
tagcagcatactctttcccct

SEQ ID NO: 6 - DNA, 1084 bp, Solanum lycopersicum
attcggttacctctctttcctatgtaactaaatgtctgctaatgtattcacaagtccaagtgatgtattcgaaattataaaatttaaggaattcttataatttgaaaaagaagtagaaaataatgtaattagctcttaacgctatgaaatttatgtaaattatataattattatgtactccttccgattcatatgacatatcttacttttaacctttacattttgttcaaaataagtaattttattgtaactaagaatgtattactattatttagtttttcaaatttacgccttcttttgataagtgggttttaacttttaacgtaaccaagaaatgatattaaatatgtactatataattaagaataattagtaaaaacaatttttaatattttaggacctaaactttttatttttttgtgcgacatgttacctaaaagatagtaaaaaaataattgccaataataaatggaataattttactagaaaataaacataggaaaagaaatatacgtaacacattaaattatatcaacggatcattaaaattcttttgtattgtctatataatactatataaaagtaaagaattctataaaattaatttgagttgacatagaaaaactgttttgggttaaattttttactagttgtgcactatttatcttcgatctataaatagatcgacatgttggaaaacactcaaaccatcctatgctataagataatatatagctacatttcttagataactagaaacctccattagcttcctattctcataagcaaatctccaatcataatttacaaactgagactcgatgtatgatcagtgatagatttaaaatttagatatcacaagtgatatgtttagatcataagggtctagaaatgcatatctaactcgatgtattctatgttgcactttgtcccgcatcacctcacaactgtaagtataaattatttcaaagagagcaggaaagtattgggtgagatattgttgacagaagatagagagcacgaataatgaggtgctaattggaagctgcaccttaattctttgtgctctctattcttctgtcat

SEQ ID NO: 7 - DNA, 1207 bp, Solanum lycopersicum
agcgaattatacagaacataattatgcaaattttgctataacatacaaatatgaattttatgtttgatatatgtgaaagttgcccattatggaattagctatgaaatttatggtaattttaagggacaattacgcggtgaagcaaacttatactacttaaatattcatcatagctatagtttgctataattaacactcgcgactaatattatacattaattatgtggcctgacttcgagtttgtataattagtcagaataaacaaatacatgttataatatacaattatctaaccgatatacataaacaatttacctctctcccactctttgccctctctcgctcgtctctctcccaatctcgttcttctcttcctccctttcccagtattgccgccactctcccaatctctctctcctctctcctccctctcccaatctctcttgccatatatacaaatacatatgtataatatacaattatataaccaatatacatatacaatgcacctctccccctctctttgccctctctcctctctctcccagtctcgcttgcctgtctcttctctataacatgtagttacagattgtaattatcaaactgtaactatgaagagtaattaaactatttttgagtgactatacgtgaaagttcctctaattttaatcaattcatcacaaatccatatctaaatgaaatgaacaaagaaaaattattattgtttagttatgaattttatcaatcactaattcacgtgaatattagggaataaaaaatgactactttggcataatctaaacttgctagtagaaatttgaagttgcaaaaagaaaaagagaagcaaaagaagtgaaagaaaaagaggcgttattgttttttactttattcagtataaagtgcgttttactcttctatttcttgtagctcacaaatcgtctttactgaccctacaaattctcttccggcaagttttcaggttcctccgaatcgctccgacgcctttgatgttcacatcttccggtagtcctgtcgcagatgactttcgcccatttatggaaccacactttctttaatttgaattctatgtggtaggacgagagtcatctgtgacaggataatggaagatcgagttatcaaaggcttattgggcgtttcctttttcatcttgagttcgtaccagattaatgcaaaaccgaagaagtag

SEQ ID NO: 8 - DNA, 1083 bp, Artificial sequence
  Solanum lycopersicum / Tomato spotted wilt tospovirus
attcggttacctctctttcctatgtaactaaatgtctgctaatgtattcacaagtccaagtgatgtattcgaaattataaaatttaaggaattcttataatttgaaaaagaagtagaaaataatgtaattagctcttaacgctatgaaatttatgtaaattatataattattatgtactccttccgattcatatgacatatcttacttttaacctttacattttgttcaaaataagtaattttattgtaactaagaatgtattactattatttagtttttcaaatttacgccttcttttgataagtgggttttaacttttaacgtaaccaagaaatgatattaaatatgtactatataattaagaataattagtaaaaacaatttttaatattttaggacctaaactttttatttttttgtgcgacatgttacctaaaagatagtaaaaaaataattgccaataataaatggaataattttactagaaaataaacataggaaaagaaatatacgtaacacattaaattatatcaacggatcattaaaattcttttgtattgtctatataatactatataaaagtaaagaattctataaaattaatttgagttgacatagaaaaactgttttgggttaaattttttactagttgtgcactatttatcttcgatctataaatagatcgacatgttggaaaacactcaaaccatcctatgctataagataatatatagctacatttcttagataactagaaacctccattagcttcctattctcataagcaaatctccaatcataatttacaaactgagactcgatgtatgatcagtgatagatttaaaatttagatatcacaagtgatatgtttagatcataagggtctagaaatgcatatctaactcgatgtattctatgttgcactttgtcccgcatcacctcacaactgtaagtataaattatttcaaagagagcaggaaagtattgggtgagatattgcagtgttgtctgtgctatatagaataatgaggtgctaattggaagctgcaccttaattcttttatatagcacagacaacactg

SEQ ID NO: 9 - DNA, 1083 bp, Artificial sequence
  Solanum lycopersicum / Tomato spotted wilt tospovirus
attcggttacctctctttcctatgtaactaaatgtctgctaatgtattcacaagtccaagtgatgtattcgaaattataaaatttaaggaattcttataatttgaaaaagaagtagaaaataatgtaattagctcttaacgctatgaaatttatgtaaattatataattattatgtactccttccgattcatatgacatatcttacttttaacctttacattttgttcaaaataagtaattttattgtaactaagaatgtattactattatttagtttttcaaatttacgccttcttttgataagtgggttttaacttttaacgtaaccaagaaatgatattaaatatgtactatataattaagaataattagtaaaaacaatttttaatattttaggacctaaactttttatttttttgtgcgacatgttacctaaaagatagtaaaaaaataattgccaataataaatggaataattttactagaaaataaacataggaaaagaaatatacgtaacacattaaattatatcaacggatcattaaaattcttttgtattgtctatataatactatataaaagtaaagaattctataaaattaatttgagttgacatagaaaaactgttttgggttaaattttttactagttgtgcactatttatcttcgatctataaatagatcgacatgttggaaaacactcaaaccatcctatgctataagataatatatagctacatttcttagataactagaaacctccattagcttcctattctcataagcaaatctccaatcataatttacaaactgagactcgatgtatgatcagtgatagatttaaaatttagatatcacaagtgatatgtttagatcataagggtctagaaatgcatatctaactcgatgtattctatgttgcactttgtcccgcatcacctcacaactgtaagtataaattatttcaaagagagcaggaaagtattgggtgagatattgatgaaatgttcggggttaaaagaataatgaggtgctaattggaagctgcaccttaattctttttttaaccccgaacatttcat

SEQ ID NO: 10 - DNA, 1083 bp, Artificial sequence
  Solanum lycopersicum / Tomato spotted wild tospovirus
attcggttacctctctttcctatgtaactaaatgtctgctaatgtattcacaagtccaagtgatgtattcgaaattataaaatttaaggaattcttataatttgaaaaagaagtagaaaataatgtaattagctcttaacgctatgaaatttatgtaaattatataattattatgtactccttccgattcatatgacatatcttacttttaacctttacattttgttcaaaataagtaattttattgtaactaagaatgtattactattatttagtttttcaaatttacgccttcttttgataagtgggttttaacttttaacgtaaccaagaaatgatattaaatatgtactatataattaagaataattagtaaaaacaatttttaatattttaggacctaaactttttatttttttgtgcgacatgttacctaaaagatagtaaaaaaataattgccaataataaatggaataattttactagaaaataaacataggaaaagaaatatacgtaacacattaaattatatcaacggatcattaaaattcttttgtattgtctatataatactatataaaagtaaagaattctataaaattaatttgagttgacatagaaaaactgttttgggttaaattttttactagttgtgcactatttatcttcgatctataaatagatcgacatgttggaaaacactcaaaccatcctatgctataagataatatatagctacatttcttagataactagaaacctccattagcttcctattctcataagcaaatctccaatcataatttacaaactgagactcgatgtatgatcagtgatagatttaaaatttagatatcacaagtgatatgtttagatcataagggtctagaaatgcatatctaactcgatgtattctatgttgcactttgtcccgcatcacctcacaactgtaagtataaattatttcaaagagagcaggaaagtattgggtgagatattgttttaaccccgaacatttcatgaataatgaggtgctaattggaagctgcaccttaattctttatgaaatgttcggggttaaaa

SEQ ID NO: 11 - DNA, 1083 bp, Artificial sequence
  Solanum lycopersicum / Tomato spotted wild tospovirus
attcggttacctctctttcctatgtaactaaatgtctgctaatgtattcacaagtccaagtgatgtattcgaaattataaaatttaaggaattcttataatttgaaaaagaagtagaaaataatgtaattagctcttaacgctatgaaatttatgtaaattatataattattatgtactccttccgattcatatgacatatcttacttttaacctttacattttgttcaaaataagtaattttattgtaactaagaatgtattactattatttagtttttcaaatttacgccttcttttgataagtgggttttaacttttaacgtaaccaagaaatgatattaaatatgtactatataattaagaataattagtaaaaacaatttttaatattttaggacctaaactttttatttttttgtgcgacatgttacctaaaagatagtaaaaaaataattgccaataataaatggaataattttactagaaaataaacataggaaaagaaatatacgtaacacattaaattatatcaacggatcattaaaattcttttgtattgtctatataatactatataaaagtaaagaattctataaaattaatttgagttgacatagaaaaactgttttgggttaaattttttactagttgtgcactatttatcttcgatctataaatagatcgacatgttggaaaacactcaaaccatcctatgctataagataatatatagctacatttcttagataactagaaacctccattagcttcctattctcataagcaaatctccaatcataatttacaaactgagactcgatgtatgatcagtgatagatttaaaatttagatatcacaagtgatatgtttagatcataagggtctagaaatgcatatctaactcgatgtattctatgttgcactttgtcccgcatcacctcacaactgtaagtataaattatttcaaagagagcaggaaagtattgggtgagatattgttcaaatgctttgcttttcaggaataatgaggtgctaattggaagctgcaccttaattctttctgaaaagcaaagcatttgaa

SEQ ID NO: 12 - DNA, 1083 bp, Artificial sequence
  Solanum lycopersicum / Tomato spotted wild tospovirus
attcggttacctctctttcctatgtaactaaatgtctgctaatgtattcacaagtccaagtgatgtattcgaaattataaaatttaaggaattcttataatttgaaaaagaagtagaaaataatgtaattagctcttaacgctatgaaatttatgtaaattatataattattatgtactccttccgattcatatgacatatcttacttttaacctttacattttgttcaaaataagtaattttattgtaactaagaatgtattactattatttagtttttcaaatttacgccttcttttgataagtgggttttaacttttaacgtaaccaagaaatgatattaaatatgtactatataattaagaataattagtaaaaacaatttttaatattttaggacctaaactttttatttttttgtgcgacatgttacctaaaagatagtaaaaaaataattgccaataataaatggaataattttactagaaaataaacataggaaaagaaatatacgtaacacattaaattatatcaacggatcattaaaattcttttgtattgtctatataatactatataaaagtaaagaattctataaaattaatttgagttgacatagaaaaactgttttgggttaaattttttactagttgtgcactatttatcttcgatctataaatagatcgacatgttggaaaacactcaaaccatcctatgctataagataatatatagctacatttcttagataactagaaacctccattagcttcctattctcataagcaaatctccaatcataatttacaaactgagactcgatgtatgatcagtgatagatttaaaatttagatatcacaagtgatatgtttagatcataagggtctagaaatgcatatctaactcgatgtattctatgttgcactttgtcccgcatcacctcacaactgtaagtataaattatttcaaagagagcaggaaagtattgggtgagatattgtagcagcatactctttcccctgaataatgaggtgctaattggaagctgcaccttaattctttaggggaaagagtatgctgcta

SEQ ID NO: 13 - DNA, 1144 bp, Artificial sequence
  Solanum lycopersicum / Tomato spotted wild tospovirus
agcgaattatacagaacataattatgcaaattttgctataacatacaaatatgaattttatgtttgatatatgtgaaagttgcccattatggaattagctatgaaatttatggtaattttaagggacaattacgcggtgaagcaaacttatactacttaaatattcatcatagctatagtttgctataattaacactcgcgactaatattatacattaattatgtggcctgacttcgagtttgtataattagtcagaataaacaaatacatgttataatatacaattatctaaccgatatacataaacaatttacctctctcccactctttgccctctctcgctcgtctctctcccaatctcgttcttctcttcctccctttcccagtattgccgccactctcccaatctctctctcctctctcctccctctcccaatctctcttgccatatatacaaatacatatgtataatatacaattatataaccaatatacatatacaatgcacctctccccctctctttgccctctctcctctctctcccagtctcgcttgcctgtctcttctctataacatgtagttacagattgtaattatcaaactgtaactatgaagagtaattaaactatttttgagtgactatacgtgaaagttcctctaattttaatcaattcatcacaaatccatatctaaatgaaatgaacaaagaaaaattattattgtttagttatgaattttatcaatcactaattcacgtgaatattagggaataaaaaatgactactttggcataatctaaacttgctagtagaaatttgaagttgcaaaaagaaaaagagaagcaaaagaagtgaaagaaaaagaggcgttattgttttttactttattcagtataaagtgcgttttactcttctatttcttgtagctcacaaatcgtctttactgaccctacaaattctcttccggcaagttttcaggttcctccgaatcgctccgacgcctttgatgttcacatcttccggtagtcccagtgttgtctgtgctatataatttatggaaccacactttctttaatttgaattctatgtggtatatatagcacagacaacactgggataatggaagatcgagttatcaaaggc

SEQ ID NO: 14 - DNA, 1144 bp, Artificial sequence
  Solanum lycopersicum / Tomato spotted wild tospovirus
agcgaattatacagaacataattatgcaaattttgctataacatacaaatatgaattttatgtttgatatatgtgaaagttgcccattatggaattagctatgaaatttatggtaattttaagggacaattacgcggtgaagcaaacttatactacttaaatattcatcatagctatagtttgctataattaacactcgcgactaatattatacattaattatgtggcctgacttcgagtttgtataattagtcagaataaacaaatacatgttataatatacaattatctaaccgatatacataaacaatttacctctctcccactctttgccctctctcgctcgtctctctcccaatctcgttcttctcttcctccctttcccagtattgccgccactctcccaatctctctctcctctctcctccctctcccaatctctcttgccatatatacaaatacatatgtataatatacaattatataaccaatatacatatacaatgcacctctccccctctctttgccctctctcctctctctcccagtctcgcttgcctgtctcttctctataacatgtagttacagattgtaattatcaaactgtaactatgaagagtaattaaactatttttgagtgactatacgtgaaagttcctctaattttaatcaattcatcacaaatccatatctaaatgaaatgaacaaagaaaaattattattgtttagttatgaattttatcaatcactaattcacgtgaatattagggaataaaaaatgactactttggcataatctaaacttgctagtagaaatttgaagttgcaaaaagaaaaagagaagcaaaagaagtgaaagaaaaagaggcgttattgttttttactttattcagtataaagtgcgttttactcttctatttcttgtagctcacaaatcgtctttactgaccctacaaattctcttccggcaagttttcaggttcctccgaatcgctccgacgcctttgatgttcacatcttccggtagtccatgaaatgttcggggttaaaaatttatggaaccacactttctttaatttgaattctatgtggtattttaaccccgaacatttcatggataatggaagatcgagttatcaaaggc

SEQ ID NO: 15 - DNA, 1144 bp, Artificial sequence
  Solanum lycopersicum / Tomato spotted wild tospovirus
agcgaattatacagaacataattatgcaaattttgctataacatacaaatatgaattttatgtttgatatatgtgaaagttgcccattatggaattagctatgaaatttatggtaattttaagggacaattacgcggtgaagcaaacttatactacttaaatattcatcatagctatagtttgctataattaacactcgcgactaatattatacattaattatgtggcctgacttcgagtttgtataattagtcagaataaacaaatacatgttataatatacaattatctaaccgatatacataaacaatttacctctctcccactctttgccctctctcgctcgtctctctcccaatctcgttcttctcttcctccctttcccagtattgccgccactctcccaatctctctctcctctctcctccctctcccaatctctcttgccatatatacaaatacatatgtataatatacaattatataaccaatatacatatacaatgcacctctccccctctctttgccctctctcctctctctcccagtctcgcttgcctgtctcttctctataacatgtagttacagattgtaattatcaaactgtaactatgaagagtaattaaactatttttgagtgactatacgtgaaagttcctctaattttaatcaattcatcacaaatccatatctaaatgaaatgaacaaagaaaaattattattgtttagttatgaattttatcaatcactaattcacgtgaatattagggaataaaaaatgactactttggcataatctaaacttgctagtagaaatttgaagttgcaaaaagaaaaagagaagcaaaagaagtgaaagaaaaagaggcgttattgttttttactttattcagtataaagtgcgttttactcttctatttcttgtagctcacaaatcgtctttactgaccctacaaattctcttccggcaagttttcaggttcctccgaatcgctccgacgcctttgatgttcacatcttccggtagtccttttaaccccgaacatttcatatttatggaaccacactttctttaatttgaattctatgtggtaatgaaatgttcggggttaaaaggataatggaagatcgagttatcaaaggc

SEQ ID NO: 16 - DNA, 1144 bp, Artificial sequence
  Solanum lycopersicum / Tomato spotted wild tospovirus
agcgaattatacagaacataattatgcaaattttgctataacatacaaatatgaattttatgtttgatatatgtgaaagttgcccattatggaattagctatgaaatttatggtaattttaagggacaattacgcggtgaagcaaacttatactacttaaatattcatcatagctatagtttgctataattaacactcgcgactaatattatacattaattatgtggcctgacttcgagtttgtataattagtcagaataaacaaatacatgttataatatacaattatctaaccgatatacataaacaatttacctctctcccactctttgccctctctcgctcgtctctctcccaatctcgttcttctcttcctccctttcccagtattgccgccactctcccaatctctctctcctctctcctccctctcccaatctctcttgccatatatacaaatacatatgtataatatacaattatataaccaatatacatatacaatgcacctctccccctctctttgccctctctcctctctctcccagtctcgcttgcctgtctcttctctataacatgtagttacagattgtaattatcaaactgtaactatgaagagtaattaaactatttttgagtgactatacgtgaaagttcctctaattttaatcaattcatcacaaatccatatctaaatgaaatgaacaaagaaaaattattattgtttagttatgaattttatcaatcactaattcacgtgaatattagggaataaaaaatgactactttggcataatctaaacttgctagtagaaatttgaagttgcaaaaagaaaaagagaagcaaaagaagtgaaagaaaaagaggcgttattgttttttactttattcagtataaagtgcgttttactcttctatttcttgtagctcacaaatcgtctttactgaccctacaaattctcttccggcaagttttcaggttcctccgaatcgctccgacgcctttgatgttcacatcttccggtagtccttcaaatgctttgcttttcagatttatggaaccacactttctttaatttgaattctatgtggtactgaaaagcaaagcatttgaaggataatggaagatcgagttatcaaaggc

SEQ ID NO: 17 - DNA, 1144 bp, Artificial sequence
  Solanum lycopersicum / Tomato spotted wild tospovirus
agcgaattatacagaacataattatgcaaattttgctataacatacaaatatgaattttatgtttgatatatgtgaaagttgcccattatggaattagctatgaaatttatggtaattttaagggacaattacgcggtgaagcaaacttatactacttaaatattcatcatagctatagtttgctataattaacactcgcgactaatattatacattaattatgtggcctgacttcgagtttgtataattagtcagaataaacaaatacatgttataatatacaattatctaaccgatatacataaacaatttacctctctcccactctttgccctctctcgctcgtctctctcccaatctcgttcttctcttcctccctttcccagtattgccgccactctcccaatctctctctcctctctcctccctctcccaatctctcttgccatatatacaaatacatatgtataatatacaattatataaccaatatacatatacaatgcacctctccccctctctttgccctctctcctctctctcccagtctcgcttgcctgtctcttctctataacatgtagttacagattgtaattatcaaactgtaactatgaagagtaattaaactatttttgagtgactatacgtgaaagttcctctaattttaatcaattcatcacaaatccatatctaaatgaaatgaacaaagaaaaattattattgtttagttatgaattttatcaatcactaattcacgtgaatattagggaataaaaaatgactactttggcataatctaaacttgctagtagaaatttgaagttgcaaaaagaaaaagagaagcaaaagaagtgaaagaaaaagaggcgttattgttttttactttattcagtataaagtgcgttttactcttctatttcttgtagctcacaaatcgtctttactgaccctacaaattctcttccggcaagttttcaggttcctccgaatcgctccgacgcctttgatgttcacatcttccggtagtcctagcagcatactctttcccctatttatggaaccacactttctttaatttgaattctatgtggtaaggggaaagagtatgctgctaggataatggaagatcgagttatcaaaggc

SEQ ID NO: 18 - DNA, 6727 bp, Artificial sequence
  Binary vector 17839
attcctgtggttggcatgcacatacaaatggacgaacggataaaccttttcacgcccttttaaatatccgattattctaataaacgctcttttctcttaggtttacccgccaatatatcctgtcaaacactgatagtttaaacgggacccggcgcgccatttaaatggtaccggtccgctggcagacaaagtggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaaaatctcagagagatagtcctagagagagaaagagagcaagtagcctagaagtaggatccatgtctccagagagaaggccagttgagattagacctgctactgcggccgatatggcagctgtttgtgatattgttaaccattatattgagacttctactgttaacttcagaactgagccacaaactcctcaagagtggattgatgatcttgagagacttcaagatagatacccttggcttgttgctgaggttgagggagttgttgctggaattgcttatgctggaccttggaaggctagaaacgcttatgattggactgttgagtctactgtttatgtttctcatagacatcaaagacttggacttggatctactctttatactcatcttcttaagtctatggaggctcaaggattcaagtctgttgttgctgttattggacttccaaacgatccatctgttagacttcatgaggctcttggatatactgctagaggaactcttagagctgctggatataagcatggaggatggcatgatgttggattctggcaaagagatttcgagcttccagctccaccaagaccagttagaccagttactcaaatttgaccatgggtcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatctgctagccctgcaggaaatttaccggtgcccgggcggccagcatggccgtatccgcaatgtgttattaagttgtctaagcgtcaatttgtttacaccacaatatatcctgccaccagccagccaacagctccccgaccggcagctcggcacaaaatcaccactcgatacaggcagcccatcagaattaattctcatgtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagaccatgagggaagcgttgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaagtagtcggcaaataaagctctagtggatctccgtacccagggatctggctcgcggcggacgcacgacgccggggcgagaccataggcgatctcctaaatcaatagtagctgtaacctcgaagcgtttcacttgtaacaacgattgagaatttttgtcataaaattgaaatacttggttcgcatttttgtcatccgcggtcagccgcaattctgacgaactgcccatttagctggagatgattgtacatccttcacgtgaaaatttctcaagcgctgtgaacaagggttcagattttagattgaaaggtgagccgttgaaacacgttcttcttgtcgatgacgacgtcgctatgcggcatcttattattgaataccttacgatccacgccttcaaagtgaccgcggtagccgacagcacccagttcacaagagtactctcttccgcgacggtcgatgtcgtggttgttgatctagatttaggtcgtgaagatgggctcgagatcgttcgtaatctggcggcaaagtctgatattccaatcataattatcagtggcgaccgccttgaggagacggataaagttgttgcactcgagctaggagcaagtgattttatcgctaagccgttcagtatcagagagtttctagcacgcattcgggttgccttgcgcgtgcgccccaacgttgtccgctccaaagaccgacggtctttttgttttactgactggacacttaatctcaggcaacgtcgcttgatgtccgaagctggcggtgaggtgaaacttacggcaggtgagttcaatcttctcctcgcgtttttagagaaaccccgcgacgttctatcgcgcgagcaacttctcattgccagtcgagtacgcgacgaggaggtttatgacaggagtatagatgttctcattttgaggctgcgccgcaaacttgaggcagatccgtcaagccctcaactgataaaaacagcaagaggtgccggttatttctttgacgcggacgtgcaggtttcgcacggggggacgatggcagcctgagccaattcccagatccccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctggctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagctctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttgatccggaatta

SEQ ID NO: 19 - DNA, 17512 bp, Artificial sequence
  Binary vector 24598
attcctgtggttggcatgcacatacaaatggacgaacggataaaccttttcacgcccttttaaatatccgattattctaataaacgctcttttctcttaggtttacccgccaatatatcctgtcaaacactgatagtttaaacgggaccgggcgccaagcttgatatcggaagtttctctcttgagggaggttgctcgtggaatgggacacatatggttgttataataaaccatttccattgtcatgagattttgaggttaatatatactttacttgttcattattttatttggtgtttgaataaatgatataaatggctcttgataatctgcattcattgagatatcaaatatttactctagagaagagtgtcatatagattgatggtccacaatcaatgaaatttttgggagacgaacatgtataaccatttgcttgaataaccttaattaaaaggtgtgattaaatgatgtttgtaacatgtagtactaaacattcataaaacacaaccaacccaagaggtattgagtattcacggctaaacaggggcataatggtaatttaaagaatgatattattttatgttaaaccctaacattggtttcggattcaacgctataaataaaaccactctcgttgctgattccatttatcgttcttattgaccctagccgctacacacttttctgcgatatctctgaggtaagcgttaacgtacccttagatcgttctttttctttttcgtctgctgatcgttgctcatattatttcgatgattgttggattcgatgctctttgttgattgatcgttctgaaaattctgatctgttgtttagattttatcgattgttaatatcaacgtttcactgcttctaaacgataatttattcatgaaactattttcccattctgatcgatcttgttttgagattttaatttgttcgattgattgttggttggtggatctatatacgagtgaacttgttgatttgcgtatttaagatgtatgtcgatttgaattgtgattgggtaattctggagtagcataacaaatccagtgttccctttttctaagggtaattctcggattgtttgctttatatctcttgaaattgccgatttgattgaatttagctcgcttagctcagatgatagagcaccacaatttttgtggtagaaatcggtttgactccgatagcggctttttactatgattgttttgtgttaaagatgattttcataatggttatatatgtctactgtttttattgattcaatatttgattgttcttttttttgcagatttgttgaccagactagtgctaaaatggataagaagtattctattggacttgatattggaaccaactctgtgggatgggctgttattactgacgagtataaggttccatctaagaagttcaaggttcttggaaacactgatagacactctattaagaagaaccttattggtgctcttcttttcgattctggagagactgctgaggctactagacttaagagaactgctagaagaagatatactagaagaaagaacagaatttgctatcttcaagagattttctctaacgagatggctaaggttgacgattctttcttccacagacttgaggagtctttccttgttgaggaggataagaagcacgagagacacccaattttcggaaacattgttgacgaggttgcttatcacgagaagtatccaactatttatcaccttagaaagaagctcgttgattctactgataaggctgatcttagacttatttatcttgctcttgctcacatgattaagttcagaggacacttccttattgagggagatcttaacccagataactctgacgttgataagctcttcattcaacttgttcaaacttataaccaacttttcgaggagaacccaattaacgcttctggagttgacgctaaggctattctttctgctagactttctaagtctagaaggcttgagaaccttattgctcaacttccaggagagaagaagaacggacttttcggaaaccttattgctctttctcttggacttactccaaacttcaagtctaacttcgatcttgctgaggacgctaagctccaactttctaaggatacttacgacgatgatcttgataaccttcttgctcaaattggagatcaatacgctgatcttttccttgctgctaagaacctttctgacgctattcttctttctgatattcttagagttaacactgagattactaaggctccactttctgcttctatgattaagagatacgacgagcaccaccaagatcttactcttcttaaggctcttgttagacaacaacttccagagaagtataaggagattttcttcgatcaatctaagaacggatacgctggatatattgacggaggagcttctcaagaggagttctataagttcattaagccaattcttgagaagatggacggaactgaggagcttcttgttaagctcaacagagaggatcttcttagaaagcaaagaactttcgataacggatctattccacaccaaattcaccttggagagcttcacgctattcttagaaggcaagaggatttctatccattccttaaggataacagagagaagattgagaagattcttactttccgtattccatattacgttggaccacttgctagaggaaactctagattcgcttggatgactagaaagtctgaggagactattactccttggaacttcgaggaggttgttgataagggagcttctgctcaatctttcattgagagaatgactaacttcgataagaaccttccaaacgagaaggttcttccaaagcactctcttctttacgagtatttcactgtttataacgagcttactaaggttaagtacgttactgagggaatgagaaagccagctttcctttctggagagcaaaagaaggctattgttgatcttcttttcaagactaacagaaaggttactgttaagcaacttaaggaggattatttcaagaagattgagtgcttcgattctgttgagatttctggagttgaggatagattcaacgcttctcttggaacttatcacgatcttcttaagattattaaggataaggatttccttgataacgaggagaacgaggatattcttgaggatattgttcttactcttactcttttcgaggatagagagatgattgaggagagacttaagacttacgctcaccttttcgacgataaggttatgaagcaacttaagagaagaagatatactggatggggtagactttctagaaagctcattaacggaattagagataagcaatctggaaagactattcttgatttccttaagtctgacggattcgctaacagaaacttcatgcaacttattcacgacgattctcttactttcaaggaggatattcaaaaggctcaagtttctggacaaggagattctcttcacgagcacattgctaaccttgctggatctccagctattaagaagggaattcttcaaactgttaaggttgttgacgagcttgttaaggttatgggtagacacaagccagagaacattgttattgagatggctagagagaaccaaactactcaaaagggacaaaagaactctagagagagaatgaagagaattgaggagggaattaaggagcttggatctcaaattcttaaggagcacccagttgagaacactcaacttcaaaacgagaagctctatctttattatcttcaaaacggaagagatatgtacgttgatcaagagcttgatattaacagactttctgattacgacgttgatcacattgttccacaatctttccttaaggacgattctattgataacaaggttcttactagatctgataagaacagaggaaagtctgataacgttccatctgaggaggttgttaagaagatgaagaactattggagacaacttcttaacgctaagctcattactcaaagaaagttcgataaccttactaaggctgagagaggaggactttctgagcttgataaggctggattcattaagagacaacttgttgagactagacaaattactaagcacgttgctcaaattcttgattctagaatgaacactaagtacgacgagaacgataagctcattagagaggttaaggttattactcttaagtctaagctcgtttctgatttcagaaaggatttccaattctataaggttagagagattaacaactatcaccacgctcacgacgcttatcttaacgctgttgttggaactgctcttattaagaagtatccaaaacttgagtctgagttcgtttacggagattataaggtttacgacgttagaaagatgattgctaagtctgagcaagagattggaaaggctactgctaagtatttcttctattctaacattatgaacttcttcaagactgagattactcttgctaacggagagattagaaagaggccacttattgagactaacggagagactggagagattgtttgggataagggaagagatttcgctactgttagaaaggttctttctatgccacaagttaacattgttaagaaaactgaggttcaaactggaggattctctaaggagtctattcttccaaagagaaactctgataagctcattgctagaaagaaggattgggacccaaagaagtacggaggattcgattctccaactgttgcttattctgttcttgttgttgctaaggttgagaagggaaagtctaagaagctcaagtctgttaaggagcttgttggaattactattatggagagatcttctttcgagaagaacccagttgatttccttgaggctaagggatataaggaggttaagaaggatcttattattaagctcccaaagtattctcttttcgagcttgagaacggaagaaagagaatgcttgcttctgctggagagcttcaaaagggaaacgagcttgctcttccatctaagtacgttaacttcctttatcttgcttctcactacgagaagctcaagggatctccagaggataacgagcaaaagcaacttttcgttgagcaacacaagcactatcttgacgagattattgagcaaatttctgagttctctaagagagttattcttgctgacgctaaccttgataaggttctttctgcttataacaagcacagagataagccaattagagagcaagctgagaacattattcaccttttcactcttactaaccttggtgctccagctgctttcaagtatttcgatactactattgatagaaagagatatacttctactaaggaggttcttgacgctactcttattcaccaatctattactggactttacgagactagaattgatctttctcaacttggaggagattcttctccaccaaagaagaagagaaaggtttcttggaaggacgcttctggatggtctagaatgtgacgtcgcgtgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggcgcgccaagcttcgttgaacaacggaaactcgacttgccttccgcacaatacatcatttcttcttagctttttttcttcttcttcgttcatacagtttttttttgtttatcagcttacattttcttgaaccgtagctttcgttttcttctttttaactttccattcggagtttttgtatcttgtttcatagtttgtcccaggattagaatgattaggcatcgaaccttcaagaatttgattgaataaaacatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgattgagaggtaaccgaatagagagttttagagctagaaatagcaagttaaaataaggctagtccgttatcaacttgaaaaagtggcaccgagtcggtgctttttttttactgatgcattgtattataagtacgttagaatgtgcaataaatatattatctatcattagaacttgaattataagtgaataatagattattttttgtaatatgaattaaaagtgtattaaacatgtattaacggtgatcaattggttaaaaaaaagtttattattaaaatgataaatctttttaatttatagtatatttatgtaagttttcacgttgagtaaatagcgaagaagttgggcccaaccaagtaaaataagaaggccgggccattacaattaagtcgtcacacaactgggcttcattgaaaaaagcgcaaaaccgattccaggcccgtgttagcatgaagactcaactcaaccagagatttctccctcatcgcttacagaaaaaagctatatgctgtttatattgcgaaatctaacagtgtagtttgaattcagggactccaatgagttttagagctagaaatagcaagttaaaataaggctagtccgttatcaacttgaaaaagtggcaccgagtcggtgctttttttttctgcagccgagacacttgtgtgattgagagaaacactaatcttgtgaggactgaagtttggtgattatttcttgtgatctgtcgacaaaaatatcaaatggggtttcttttacaaattatttacctaaatgaatctgttttgaaaatatttactccatgggtctatttttttattacaaagcgtctccctgaagggcgcgttccccgtgaaagtgacacgtggcaggacttgggacgtgccctgcgtacaggcgcgatagttagtgttgttacagcaggcgcatcgggtcgtgttggggaccaaggtacgacaggtcgcgctggggacccagacacgacccaattgggtcgcactttatttaatattttttatattttgtatattgtttttatttaatatatttttatattattttatttaatttttttatattttatataatagtttctatattaaataaattcttagcattatgtatgattttaaagtcataaataattttttatattgtttttatttactatattttttatattttatttaatatttatatattaaataaatccttcatattagaaaaaataaagaaaatattaaataaaatataaaatataaaaaagtaaaaaatattaaataaaataatataaaaaatattataaaaacaatataaaaaatataaaaatatttaataaaataataaaaaaaatattattttaaataaaattatttatgactttaaactctaaagttgaattttaaaaaaatataatttttttacgattttagtaaaaaaaaaatacaagccgcacaatacaagtcgccttctcaaacccttcctcacgacattctcggaccttatgacaccgtcaccaaaacaatgatccacgcgatattaggcgcgtgcaaatcactctaatccgaaactagtagacatgggaagcacgagctatacgcgagcgtttcaattgccgccacgaaagcagagaaggccagaaacggaaccacggtaaaatggtaagggtattttcgtaaacagaagaaaagagttgtagctataaataaaccctctaacccacggcgcactatttctcttcactccttcgttcactcttcttctcttgcggctagggttttagcgcagcttcttctaggttcgttctcttccgccgctctatggattttaaaccttcgaatcatgtttattccattgaattatgttgcttgcagtttatattttctgaatctgtagttgttgtcttcaatttatcctatgctttatagatcaatcttttgtgtgtgtagtacgtaatttttgttctttttgcttttcgttcaagttgttgggaataatcggggtatcatgttttgatattgtttgttttcttttttgactgcttaataatttttaagttggttttggttttggggttttatgtgcttgttatattcaaatctttggatccagatcttacaaaagttttgggtttaaggatgtttttggctgatgatgaatagatctataaactgttccttttaatcgattcaagcttaggattttactaggcttttgcgaataaatacgtgacagtaagctaattatgtcctttttttgtctcaatcatatctgtctgggtgtgccataatttgtgatatgtctatctggtagaatcttgtgttttatgctttacgatttggtatacctgtttttgaacttgttgtatgatgggtatttagatcaccctatcttttttatgcttctggaagttttatgtaaatgtcgaatatcttaatgttgttgaacttataatgttgtgttgatgtatgtatgatggttttgacaacttttttcactggttctgaaagttttatgtaaattgcaaatatgttaatgttgttgaacttattttttttccttcgatgttgttttgatgtatgtatgatggttttcaccgtagtttctatggctaatatcttaatgttgttgagcttatttttttccttatatgttgtgttgatgtatgtatgatggttttgacaacttttttagtttctttgcagatttaaggaagatcgatggcgcaagttagcagaatctgcaatggtgtgcagaacccatctcttatctccaatctctcgaaatccagtcaacgcaaatctcccttatcggtttctctgaagacgcagcagcatccacgagcttatccgatttcgtcgtcgtggggattgaagaagagtgggatgacgttaattggctctgagcttcgtcctcttaaggtcatgtcttctgtttccacggcgtgcatgagggaagcgttgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaagtagtcggcaaataatgagctcatctagctagagctttcgttcgtatcatcggtttcgacaacgttcgtcaagttcaatgcatcagtttcattgcgcacacaccagaatcctactgagtttgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttgccaattgattgacaacatgcatcaatcccgggcggccagcatggccgtatccggatgtcatattccctatctgatcgtgagaggtaaccgaatagagagggtttcctatgtaactaaatgtctgctaatgtattcacaagtccaagtgatgtattcgaaattataaaatttaaggaattcttataatttgaaaaagaagtagaaaataatgtaattagctcttaacgctatgaaatttatgtaaattatataattattatgtactccttccgattcatatgacatatcttacttttaacctttacattttgttcaaaataagtaattttattgtaactaagaatgtattactattatttagtttttcaaatttacgccttcttttgataagtgggttttaacttttaacgtaaccaagaaatgatattaaatatgtactatataattaagaataattagtaaaaacaatttttaatattttaggacctaaactttttatttttttgtgcgacatgttacctaaaagatagtaaaaaaataattgccaataataaatggaataattttactagaaaataaacataggaaaagaaatatacgtaacacattaaattatatcaacggatcattaaaattcttttgtattgtctatataatactatataaaagtaaagaattctataaaattaatttgagttgacatagaaaaactgttttgggttaaattttttactagttgtgcactatttatcttcgatctataaatagatcgacatgttggaaaacactcaaaccatcctatgctataagataatatatagctacatttcttagataactagaaacctccattagcttcctattctcataagcaaatctccaatcataatttacaaactgagactcgatgtatgatcagtgatagatttaaaatttagatatcacaagtgatatgtttagatcataagggtctagaaatgcatatctaactcgatgtattctatgttgcactttgtcccgcatcacctcacaactgtaagtataaattatttcaaagagagcaggaaagtattgggtgagatattgttttaaccccgaacatttcatgaataatgaggtgctaattggaagctgcaccttaattctttatgaaatgttcggggttaaaacatcttcagtccctccccgaccctctctaccttaatttatttctacgtttattgtatttaaatttccctatatgtcctcctttatcttcaaaatcgaaaaatgaagttatattaatttgtttagtgtaacttaactcttgaccatgctgcttccgatcaagaaagggttttattgatgatagttaattagttacgttagcttataaattacaaacttctagaaaagttctatgactatttattgatacaattcacatcgatgtaatgaaagtgaaaaattcataataattatagaaaatcatgaataatcgattcgtttgacaactataatatagtctcacaaaatcttttatctttgccttaaattacatctttgccttaaattacatcaaaaaatgatttgtaaactttattatgatcacgaattcagggactccaatgaaggcatcattaagaagtgtatccatagtttcttgtactaatttcgtatccgcaatgtgttattaagttgtctaagcgtcaatttgtttacaccacaatatatcctgccaccagccagccaacagctccccgaccggcagctcggcacaaaatcaccactcgatacaggcagcccatcagaattaattctcatgtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagaccatgagggaagcgttgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaagtagtcggcaaataaagctctagtggatctccgtacccagggatctggctcgcggcggacgcacgacgccggggcgagaccataggcgatctcctaaatcaatagtagctgtaacctcgaagcgtttcacttgtaacaacgattgagaatttttgtcataaaattgaaatacttggttcgcatttttgtcatccgcggtcagccgcaattctgacgaactgcccatttagctggagatgattgtacatccttcacgtgaaaatttctcaagcgctgtgaacaagggttcagattttagattgaaaggtgagccgttgaaacacgttcttcttgtcgatgacgacgtcgctatgcggcatcttattattgaataccttacgatccacgccttcaaagtgaccgcggtagccgacagcacccagttcacaagagtactctcttccgcgacggtcgatgtcgtggttgttgatctagatttaggtcgtgaagatgggctcgagatcgttcgtaatctggcggcaaagtctgatattccaatcataattatcagtggcgaccgccttgaggagacggataaagttgttgcactcgagctaggagcaagtgattttatcgctaagccgttcagtatcagagagtttctagcacgcattcgggttgccttgcgcgtgcgccccaacgttgtccgctccaaagaccgacggtctttttgttttactgactggacacttaatctcaggcaacgtcgcttgatgtccgaagctggcggtgaggtgaaacttacggcaggtgagttcaatcttctcctcgcgtttttagagaaaccccgcgacgttctatcgcgcgagcaacttctcattgccagtcgagtacgcgacgaggaggtttatgacaggagtatagatgttctcattttgaggctgcgccgcaaacttgaggcagatccgtcaagccctcaactgataaaaacagcaagaggtgccggttatttctttgacgcggacgtgcaggtttcgcacggggggacgatggcagcctgagccaattcccagatccccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctggctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagctctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttgatccggaatta

SEQ ID NO: 20 - DNA, 20 bp, Artificial sequence
  gRNA sequence
gagaggtaaccgaatagaga

SEQ ID NO: 21 - DNA, 20 bp, Artificial sequence
  gRNA sequence
gaattcagggactccaatga

SEQ ID NO: 22 - DNA, 21 bp, Tomato spotted wilt tospovirus
tatatagcacagacaacactg

SEQ ID NO: 23 - DNA, 21 bp, Tomato spotted wilt tospovirus
ctgaaaagcaaagcatttgaa

SEQ ID NO: 24 - DNA, 21 bp, Tomato spotted wilt tospovirus
aggggaaagagtatgctgcta